(19) United States
(12) Patent Application Publication
(10) Pub. No.: US 2026/0022422 A1
(43) Pub. Date: Jan. 22, 2026

(54) METHOD OF DETECTING TUMOUR RECURRENCE

(71) Applicants: Natera, Inc., San Carlos, CA (US); UCL BUSINESS PLC, London (GB)

(72) Inventors: Robert Charles SWANTON, London (GB); Christopher ABBOSH, Herts (GB)

(73) Assignee: Natera, Inc., San Carlos, CA (US)

(21) Appl. No.: 19/176,873

(22) Filed: Apr. 11, 2025

Related U.S. Application Data
(63) Continuation of application No. 16/347,134, filed on May 2, 2019, now Pat. No. 12,460,264.

(30) Foreign Application Priority Data
Nov. 2, 2016 (GB) 1618485.5

(51) Int. Cl. C12Q 1/6886; C12Q 1/686

(52) U.S. Cl. CPC C12Q 1/6886; C12Q 1/686; C12Q 2600/106; C12Q 2600/118; C12Q 2600/156; C12Q 2600/16

(57) ABSTRACT

A system and method for determining the genetic data for one or a small set of cells, or from fragmentary DNA, where a limited quantity of genetic data is available, and also for predicting likely phenotypic outcomes using mathematical models and given genetic, phenotypic and/or clinical data of an individual, and also relevant aggregated medical data consisting of genotypic, phenotypic, and/or clinical data from germane patient subpopulations. Genetic data for the target individual is acquired and amplified using known methods, and poorly measured base pairs, missing alleles and missing regions are reconstructed using expected similarities between the target genome and the genome of genetically related subjects.

### FIELD OF THE INVENTION

The present invention relates to a subject-specific method for detecting recurrence of a tumour in a subject.

### BACKGROUND TO THE INVENTION

Cancer is a leading cause of death worldwide, with lung cancer being the leading cause of cancer-related mortality. Non-small cell lung cancer (NSCLC) is the most common histological subtype, accounting for 85% to 90% of lung cancer cases. The majority of patients with NSCLC present with locally advanced or metastatic disease and have a poor prognosis. Even if identified at a resectable stage, up to 30% to 50% of patients will experience disease relapse in the post-operative setting.

Up to 40% of patients who undergo surgical resection of a non-small cell lung cancer, experience relapse of their cancer. Currently some patients who undergo surgery are offered postsurgical (adjuvant) chemotherapy or radiotherapy to reduce the risk of their lung cancer coming back by 5-10%. This treatment is offered based on whether a patient has pathological features consistent with an aggressive tumour (i.e. a large tumour, lymph gland involvement) and whether a patient is fit enough to receive chemotherapy treatment.

Following chemotherapy patients are put on “surveillance”. They return to clinic every 3-6 months to be assessed clinically for signs of lung cancer relapse and they also receive a chest X-ray or CT scan. If there are imaging or clinical features consistent with relapse of their lung cancer, they will be formally investigated for relapse. If relapsed lung cancer is confirmed treatment depends on where the relapsed lung cancer has spread to. If the lung cancer has relapsed in a single site, then “radical” treatment with radiotherapy or surgery can be carried out to try to achieve a cure for the patient. If the cancer relapse in multiple sites, then treatment with chemotherapy is usually offered and patients are unable to be cured. If this is the case, then prognosis is usually limited to a year to 18 months.

Cell free DNA (cfDNA) can be detected in the plasma and serum of patients with advanced cancer. Multiple approaches to cfDNA profiling are in the process of being established as clinical diagnostic tools. However, in early-stage lung cancer, approaches to cfDNA profiling are less successful and typically use generic gene panels to interrogate a plasma sample.

### SUMMARY OF THE INVENTION

The present invention describes a “bespoke” approach to tracking tumour recurrence and the nature of the metastatic subclone(s) in a non-invasive fashion by detecting patient-specific clonal and/or subclonal mutations in the cfDNA from the patient's plasma. Through modelling of a patient's tumour “phylogenetic tree” the method generates a bespoke biomarker profile unique to an individual patient. Clonal mutations, by their very nature, are present in every cell within a tumour. By incorporating clonal mutations into a panel this approach increases the sensitivity of plasma variant detection. Tracking a plasma profile of a patient's tumour “phylogenetic tree” allows identification of the clone/subclone(s) within a tumour that is/are responsible for seeding relapsed disease, opening opportunities for interventional therapeutic opportunities before macroscopic disease is evident on imaging and monitoring response to such therapies by tracking the decline of clonal and subclonal variants.

Thus, by tracking patient-specific variants and by selecting variants that represent clonal variant from the trunk of a patient's phylogenetic tree, rather than relying on a generic panel of markers that might or might not be present in any individual patient, this methodology advantageously increases the sensitivity and specificity by which plasma variants are identified.

Thus, the invention relates to a method for cfDNA profiling in a tumour, for example lung cancer, involving tracking a patient's disease progression and/or recurrence to aid robust treatment regimens. This approach finds particular utility in tracking recurrence of lung cancer, especially non-small cell lung cancer, after surgical resection, in the adjuvant setting. Early detection of recurrence using this method could prompt initiation of therapy (including immunotherapy) or change in therapy, using the same approach to track the response of such residual disease to therapy. This approach will also have utility in cancers other than those in the lung and/or that have been removed in other ways.

Thus, in a first aspect of the present invention there is provided a subject-specific method for detecting recurrence of a tumour in a subject, comprising:
(a) sequencing all or part of the genome or exome of a tumour of a subject to define clonal and/or subclonal mutations in said tumour;
(b) defining a set of reagents that will detect the presence of DNA from said tumour via the presence of said clonal and/or subclonal mutations;
using said set of reagents, analysing a sample comprising DNA from said tumour obtained from the subject subsequent to the tumour removal to determine whether or not said tumour has recurred by detection of said clonal and/or subclonal mutations in the sample.

In a further aspect of the invention, there is provided a subject-specific method of defining a set of reagents to detect recurrence of a tumour in the subject comprising:
(a) sequencing all or part of the genome or exome of a tumour of the subject;
(b) defining clonal and/or subclonal mutations in said tumour; and defining a set of reagents that will detect the presence of said clonal and/or subclonal mutations in a sample from said subject.

In another aspect of the invention, there is provided a subject-specific method for detecting recurrence of a tumour in a subject comprising analysing, with a subject-specific set of reagents that will detect the presence of clonal and/or subclonal mutations from said tumour, DNA in a sample obtained from the subject subsequent to removal of a tumour to determine whether or not said tumour has recurred by detection of said clonal and/or subclonal mutations from the tumour in the sample.

In yet another aspect of the invention, there is provided a set of subject-specific detection reagents for the detection of clonal and/or subclonal mutations from a tumour in the cfDNA of the subject, obtained or obtainable by a method of the invention.

In a further aspect of the invention there is provided, a method of treating a subject comprising performing a method of the invention wherein a treatment is chosen for the patient based on the clonal and/or subclonal mutations detected in the sample from the subject.

### BRIEF DESCRIPTION OF THE FIGURES

FIG. 1. Workflow diagram demonstrating target selection, assay design and plasma PCR.

FIG. 2. Measured cfDNA concentration, tumour stage. Each data point represents one plasma sample.

FIG. 3. Validation of multiplex PCR reactions using DNA from patient tumour samples. Samples that showed good correlation between tissue VAF measurements. Each sample is shown in a separate box, and the VAF data points are coloured by tissue subsection.

FIG. 4. SNVs detected and not detected in plasma for each sample.

FIG. 5. SNV detection (left) and sample detection (right) in plasma by tumour stage.

FIG. 6. Plasma VAF as a function of tumour stage and SNV clonality.

FIG. 7. Number of SNVs detected in plasma by histological type.

FIG. 8. Number of SNVs detected in plasma from each sample as a function of the cfDNA input amount.

FIG. 9. Detection of SNVs in plasma was correlated with their VAF in tumour.

FIG. 10A. Depth of read histogram as a function of the resulting call.

FIG. 10B. Estimated limit of detection of the assays as a function of the resulting call. In each panel, the left group shows the assays that did not detect the expected plasma SNV, and the right group shows the assay that detected the expected plasma SNV.

FIG. 11A. Assessment of SNVs detected in plasma for recapitulating tumour phylogeny trees from cfDNA data. Samples with more than 10 plasma SNVs are shown. Number of branches detected in plasma with valid estimated cancer cell fraction.

FIG. 11B. Assessment of SNVs detected in plasma for recapitulating tumour phylogeny trees from cfDNA data. Samples with more than 10 plasma SNVs are shown. Percentage of pairs of SNVs for which their plasma VAF values follow the tumour phylogeny tree structure.

FIG. 12. Top panel—total number of SNVs targeted by primers, per sample, sorted by driver category. Bottom panel—total number of SNVs targeted by primers, per sample, sorted by clonality.

FIG. 13. Bar chart demonstrating the number of clonal and subclonal variants detected per patient sorted based on histological subtype, squamous cell carcinoma (LUSC) and adenocarcinoma (LUAD). Filled bars demonstrate detected variants, unfilled bars demonstrate the number of variants probed for.

FIGS. 14A-14D. Detected lung adenocarcinomas (LUADs) are more mitotically active (FIGS. 14A and 14B), and genomically unstable (FIG. 14C) that undetected LUADs. Detected LUADs are largely the solid histological subtype (FIG. 14D).

FIGS. 15A-15D. Clonal SNVs have higher plasma variant allele frequencies than subclonal SNVs (FIGS. 15A and 15B), mean clonal variant allele frequency (per patient) correlates with tumour volume (FIG. 15C) (Spearman's Rank Rₛ(42)=0.60, P<0.001). PET CT from outlier in FIG. 15C demonstrating only a rim of PET avid tumour (FIG. 15D).

FIGS. 16A-16B. The mean normalized variant allele frequency (VAF) of called mutations correlates with mutation copy number as determined by whole exome sequencing, the mean normalized VAF of called mutations correlates with mean subclonal cluster Cancer Cell Fraction taken from all involved regions as identified by multi-region sequencing (an inference of subclone size).

FIGS. 17A-17M. Time course graphs demonstrating detection of clonal SNVs in plasma in advance of clinical relapse in patients with lung squamous cell carcinomas (LUSC) and lung adenocarcinomas (LUADs) who suffered relapse of their disease within this study.

FIGS. 18A-18L. Time course graphs demonstrating detection of clonal SNVs in plasma in advance of clinical relapse in patients with lung squamous cell carcinomas (LUSC) and lung adenocarcinomas (LUADs) who did not suffer relapse of their disease within the time course of this study (control cases).

FIGS. 19A-19E. Top row of “trees” represent phylogenetic trees constructed from the exome data. The bottom row of trees represent plasma phylogenetic trees. These figures demonstrate how plasma genotyping can be used to represent the subclonal structure of a tumour.

FIGS. 20A-20B. Tracking the subclonal structure of a tumour through time. In LTX019 a particular subclonal cluster was detected at the point of lung cancer relapse. This subclonal cluster contains CD44 which is associated with metastatic niche formation. In LTX038 only one phylogenetic branch is detected at relapse—suggesting that the relapse clone arose from this branch.

FIG. 21. CONSORT diagram demonstrating patient involved in this cfDNA analysis and why 4 patients from the exome study were removed fom the cfDNA analysis.

FIG. 22. Correlation of prevalence of mutational signatures and different mutation categories in LUAD and LUSC tumours. Significance from Spearman's Rank is indicated. To indicate that mutational signatures can be used to infer the clonality of a mutation.

### DETAILED DESCRIPTION OF THE INVENTION

### Subject-Specific Method

The present invention relates to a subject-specific method for detecting recurrence of a tumour in a subject. The method is subject-specific insofar as the method is tailored to the patient. The method is carried out for a particular patient who has been diagnosed with a tumour. Subject specific can also be referred to as patient specific. The clonal and/or subclonal mutations identified in the tumour, that are used to determine whether the tumour has recurred in the patient, are mutations previously determined to be present in the tumour resected or biopsied from the individual subject.

In a preferred embodiment of the present invention, the subject described herein is a mammal, preferably a human, cat, dog, horse, donkey, sheep, pig, goat, cow, mouse, rat, rabbit or guinea pig. Most preferably the subject is a human.

The method of the invention detects the recurrence of a tumour in the specific subject or patient by detecting the presence and/or rise of clonal and/or subclonal mutations that are characteristic of the patient's tumour in the cell free DNA (cfDNA). It will be appreciated that the term “clonal mutation” may include an early clonal mutation(s) and/or a clonal mutation(s). The clonal and/or subclonal mutations are characteristic of the specific tumour from the individual patient. The clonal and/or subclonal mutations characteristic of the patient's tumour are defined by sequencing all or part of the whole genome and/or exome of DNA from the tumour, typically after the tumour has been resected from the patient. Using a set of reagents designed or defined to detect the presence of DNA from the tumour via the presence of the specific clonal and/or subclonal mutations identified for the specific subject of interest, the presence and/or rise of clonal and/or subclonal mutations in cfDNA obtained from the patient is analysed.

The presence and/or rise of clonal and/or subclonal mutations in the cfDNA from the patient characteristic of the tumour indicates whether the tumour has recurred. The presence and/or rise of clonal mutations in the cfDNA from a specific patient characteristic of the tumour may indicate relapse of the tumour.

In a preferred embodiment, the presence and/or rise of clonal mutations in the cfDNA from the patient characteristic of the tumour indicates whether the tumour has recurred. The presence and/or rise of clonal mutations in the cfDNA from a specific patient characteristic of the tumour may indicate relapse of the tumour.

In a preferred embodiment, the presence and/or rise of early clonal mutations in the cfDNA from the patient characteristic of the tumour indicates whether the tumour has recurred. The presence and/or rise of early clonal mutations in the cfDNA from a specific patient characteristic of the tumour may indicate relapse of the tumour.

The presence and/or rise of clonal and/or subclonal mutations in the cfDNA from the patient characteristic of the tumour may be used to determine which part or parts of the tumour is seeding relapse of the tumour in the patient. The presence and/or rise of subclonal mutations in cfDNA characteristic of the tumour may determine the part or parts of the tumour seeding relapse of the tumour.

In a preferred embodiment, the presence and/or rise of subclonal mutations in the cfDNA from the patient characteristic of the tumour may be used to determine which part or parts of the tumour is seeding relapse of the tumour in the patient. The presence and/or rise of subclonal mutations in cfDNA characteristic of the tumour may determine the part or parts of the tumour seeding relapse of the tumour.

### Tumour Type

The subject-specific method of the present invention is directed to detecting recurrence of a tumour. The tumour may be any solid or non-solid tumour. The tumour may be, for example, bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukaemia, lymphomas, pancreatic cancer, hepatobiliary tumours, germ cell cancers, prostate cancer, head and neck cancers, thyroid cancer and sarcomas.

In a preferred embodiment of the present invention, the tumour type is lung cancer, preferably non-small cell lung cancer. In a further aspect of the invention, the non-small cell lung cancer is a squamous cell tumour, an adenocarcinoma or a large cell carcinoma. In a preferred embodiment, the non-small cell lung cancer is squamous cell carcinoma.

The tumour may be removed from the patient by a number of different techniques known to the person skilled in the art. For example, the tumour may be removed or eliminated from the patient by surgical resection, endoscopic resection, chemotherapy and/or radiotherapy.

### Tumour DNA

The method of the present invention comprises the step of determining the mutations present in cancer cells isolated from a tumour or subsection of a tumour. Isolation of biopsies and samples from tumours is common practice in the art and may be performed according to any suitable method and such methods will be known to one skilled in the art.

The tumour sample may be a blood sample. For example the blood sample may comprise cfDNA, circulating tumour DNA, circulating tumour cells or exosomes comprising tumour DNA.

In a preferred embodiment, early clonal, clonal and/or subclonal mutations in the tumour are defined from one or more tumour subsections. Early clonal, clonal and/or subclonal mutations in the tumour may be defined from at least one, at least two, at least three, at least four, at least five, at least six, at least seven, at least eight, at least nine, at least or up to ten subsections of the tumour.

In a further embodiment, clonal and/or subclonal mutations in the tumour are defined from a single region. In yet a further embodiment, early clonal mutations, i.e. clonal alterations that occur prior to genome doubling, are defined from a single region.

A single region encompasses a tumour biopsy, a subsection of a tumour, a whole tumour, a selected or single region of the exome, or selected or single region of the whole genome sequence. A single region of the exome or of the whole genome sequence may be selected on the region which encodes for early clonal mutations that occur pre-genome doubling that have undergone copy number amplifications. A single region of the exome or of the whole genome sequence may be selected on the region which encodes for common clonal somatic and early amplification events (see, for example, Table 1) and/or the likely “mutation signature” of a clonal and/or subclonal mutation (see, for example, Table 2). A single region of the exome or of the whole genome sequence may be selected by extrapolating likely clonal mutations characteristic of the tumour in the patient from the data relating to common clonal somatic and early amplification events (see, for example, Table 1) and/or the likely “mutation signature” of a clonal and/or subclonal mutation (see, for example, Table 2).

### Clonal and Subclonal Mutations

A ‘mutation’ refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual.

References herein to “essentially all” are intended to encompass the majority of tumour cells in a subject. For example, this may comprise 60-100% of cells, e.g. 60, 61, 62, 63, 64, 65, 66, 67, 68, 69, 70, 71, 72, 73, 74, 75, 76, 77, 78, 79 80, 81, 82, 83, 84, 85, 86, 87, 88, 89, 90, 91, 92, 93, 94, 95, 96, 97, 98, 99 or 100% of tumour cells in a subject.

Different regions of tumours may be morphologically distinct. In addition, intratumour mutational heterogeneity may occur and can be associated with differences in tumour prognosis and the potential ability of tumour cells to escape immune therapies targeting mutations which are not present in all or most tumour cells.

Intratumour heterogeneity causes expression of tumour mutations in different regions of the tumour and between different cells of the tumour. Within a tumour, certain mutations are expressed in all regions and essentially all cells of the tumour whilst other mutations are only expressed in a subset of tumour regions and cells.

A “truncal” or “clonal” mutation is a mutation which is expressed effectively throughout a tumour and encoded within essentially every tumour cell. A “branch” or “subclonal” mutation is a mutation which is expressed in a subset or a proportion of cells or regions in a tumour.

‘Present throughout a tumour’, ‘expressed effectively throughout a tumour’ and ‘encoded within essentially every tumour cell’ may mean that the truncal mutation is expressed in all regions of the tumour from which samples are analysed.

It will be appreciated that a determination of a mutation is ‘encoded within essentially every tumour cell’ refers to a statistical calculation and is therefore subject to statistical analysis and thresholds.

A determination that a truncal mutation is ‘expressed effectively throughout a tumour’ refers to a statistical calculation and is therefore subject to statistical analysis and thresholds.

“Expressed effectively in essentially every tumour cell or essentially all tumour cells means” that the mutation is present in all tumour cells analysed in a sample, as determined using appropriate statistical methods.

By way of example, the cancer cell fraction (CCF), describing the proportion of cancer cells that harbour a mutation may be used to determine whether mutations are truncal or branched. For example, the cancer cell fraction may be determined by integrating variant allele frequencies with copy numbers and purity estimates as described by Landau et al. (Cell. 2013 Feb. 14;152(4):714-26).

In brief, CCF values are calculated for all mutations identified within each and every tumour region analysed. If only one region is used (i.e. only a single sample), only one set of CCF values will be obtained. This will provide information as to which mutations are present in all tumour cells within that tumour region, and will thereby provide an indication if the mutation is truncal or branched. All sub clonal mutations (i.e. CCF<1) in a tumour region are determined to be branched, whilst clonal mutations with a CCF=1 are determined to be truncal.

As stated, determining a truncal mutation is subject to statistical analysis and threshold. As such, a mutation may be identified as truncal if it is determined to have 5 a CCF 95% confidence interval >=0.75, for example 0.80, 0.85, 0.90, 0.95, 1.00 or >1.00. Conversely, a mutation may be identified as branched if it is determined to have a CCF 95% confidence interval <=0.75, for example 0.70, 0.65, 0.60, 0.55, 0.50, 0.45, 0.40, 0.35, 0.30, 0.25, 0.20, 0.15, 0.10, 0.05, 0.01 in any sample analysed. It will be appreciated that the accuracy of a method for identifying truncal mutations is increased by identifying clonal mutations for more than one sample isolated from the tumour.

The mutation may be a single nucleotide variant (SNV), multiple nucleotide variants, a deletion mutation, an insertion mutation, a translocation, a missense mutation or a splice site mutation resulting in a change in the amino acid sequence (coding mutation).

In a preferred embodiment, the clonal and/or subclonal mutations are single nucleotide variants (SNVs).

In some embodiments of the present invention, the clonal mutations are early clonal mutations.

It is known that genome doubling can occur in cancer cells. A mutation which occurs before a genome doubling event will therefore be present in a cancer cell at twice the relative copy number of a mutation which occurred after the doubling event. Early clonal mutations are those that arise in the tumour before genome doubling. Early clonal mutations encompass mutations which occur before a genome doubling event and are present in a cancer cell at twice the relative copy number of a mutation which occurred after the doubling event, i.e. mutations that occur pre-genome doubling and that have undergone copy number amplifications. It will be appreciated that the term “clonal” may include early clonal mutations and/or clonal mutations. Early clonal mutations may be present in chromosomal regions or in specific genes. Early clonal mutations may be somatic SNVs in genes classified as being high-confidence pre-genome doubling events and/or somatic SNVs in chromosomal regions classified as being involved in high-confidence early (pre-genome doubling) amplification events. Early clonal mutations may be detected in one or more of the genes and/or chromosomal regions of Table 1.

The presence and/or rise of early clonal mutations and/or clonal mutations in cfDNA that are characteristic of a tumour in a specific patient indicates the relapse of the tumour. Thus, detecting the presence of these clonal and/or early clonal mutations in cfDNA detects whether the tumour has relapsed. Early clonal mutations encompass mutations that occur pre-genome doubling that have undergone copy number amplifications and that have the highest variant allele frequency in plasma.

A mutation profile which includes early clonal, clonal and/or subclonal mutations of the tumour may be used to reconstruct the phylogenetic tree of each tumour from each patient. It will be appreciated that the phylogenetic tree of each tumour represents how the tumour has evolved. The evolution of the tumour is coded in the tree and provides important biological information about the genetic diversity of heterogeneity of the cancer and early clonal, clonal and/or subclonal mutation composition. Mutations from a tumour may be assembled into a phylogenetic tree defining the early clonal, clonal and/or subclonal mutation profile of the tumour. Such a phylogenetic tree will be specific for the tumour and specific for the patient from which the tumour has been resected.

The phylogenetic tree characterises the early clonal, clonal and/or subclonal mutations in a tumour specific for a patient.

The early clonal and/or clonal mutations or clones are in the “trunk” of the tree. Clonal mutations are relapse indicators and detecting their presence and/or rise in a sample, for example cfDNA, is indicative of relapse of the tumour. Early clonal mutations have high variant allele frequencies in cfDNA and their presence and/or rise represents a sensitive method of predicting or indicating relapse of the tumour post-surgery.

The subclonal mutations are in the “branches” of the tree. Detection of the subclonal mutations or subclones allows for the identification of the branches seeding relapse of the tumour. Identifying the branches seeding relapse may have implications in patient specific treatment options.

### Barcodes

In some embodiments, a patient-specific “barcode” is generated from the early clonal, clonal and/or subclonal mutations as exemplified in Examples 8 and 9. A patient-specific barcode comprises or consists of SNVs and/or insertion and/or deletion mutations that are characteristic of the tumour in the specific patient that can be clonal or subclonal in origin to depict the tumour phylogenetic tree. Detection of the SNVs of the patient-specific barcode indicates whether the tumour in the specific patient has relapsed. A barcode is specific for the tumour in the specific patient. Thus, the barcode is unique to each patient. The barcode may also be referred to as a personalised barcode.

The patient-specific barcode may comprise one or more mutations. The patient- specific barcode may comprise one or more SNVs and/or insertion and/or deletion mutations. The patient-specific barcode may comprise at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 SNVs and/or insertion and/or deletion mutations characteristic of the tumour of the specific patient.

The SNVs of the barcode may be early clonal mutations, clonal mutations and/or subclonal mutations and/or insertion and/or deletion mutations. A patient specific barcode may therefore comprise or consist of early clonal mutations, clonal mutations and/or subclonal mutations and/or insertion and/or deletion mutations. A patient specific barcode may also comprise early clonal mutations only. A patient specific barcode may also comprise SNVs arising from early clonal driver genes and/or early clonal amplification chromosome arms (Table 1) and/or the mutation signature (Table 2). In a preferred embodiment, the SNVs that comprise or consist the patient-specific barcode are those that give a high sensitivity and high resolution for detecting relapse of the tumour.

A patient-specific barcode may comprise early clonal mutations, clonal mutations and/or subclonal mutations derived from a single region. A single region is described above. A single region patient specific barcode may therefore comprise or consist of early clonal mutations, clonal mutations and/or subclonal mutations and/or insertion and/or deletion mutations. A single region patient specific barcode may also comprise early clonal mutations only. A single region patient specific barcode may also comprise SNVs arising from early clonal driver genes and/or early clonal amplification chromosome arms (Table 1) and/or the mutation signature (Table 2). In a preferred embodiment, the SNVs that comprise or consist the single region patient-specific barcode are those that give a high sensitivity and high resolution for detecting relapse of the tumour.

### Cell Free DNA (cfDNA)

The invention relates to a subject-specific method of detecting recurrence of a tumour in a subject wherein the clonal and/or subclonal mutations are identified in a sample. The sample may be a body fluid. Body fluid refers to any body fluid in which cfDNA, exosome derived tumour DNA, circulating tumour DNA and/or circulating tumour cells may be present including, without limitation, whole blood, serum, plasma, bone marrow, cerebral spinal fluid, peritoneal/pleural fluid, lymph fluid, ascite, serous fluid, sputum, lacrimal fluid, stool, and urine. Typically, the body fluid to be analysed according to the invention is derived from a sample of whole blood (i.e., a blood sample comprising both blood cells and plasma). Preferably, the body fluid is plasma or serum. The sample may comprise cfDNA, circulating tumour DNA, circulating tumour cells or exosomes comprising tumour DNA. It will be appreciated that exosome derived tumour DNA refers to exosomes that are released by the tumour cells and contain tumour DNA. The tumour DNA is contained within the exosome and derived from the exosome.

The sample may be obtained from the specific patient by any non-invasive method. A non-invasive method encompasses any method that does not invade the body or cut open the body. Such methods include, but are not limited to, venepuncture, methods to obtain a liquid biopsy, lumbar puncture, pleural fluid aspiration, pericardial fluid aspiration and ascitic fluid aspiration.

In a preferred embodiment, the sample comprises cfDNA. Cell-free DNA or “cfDNA” herein refers to DNA that exists outside a cell in a subject or the isolated form of such DNA, typically in a body fluid. Unless otherwise indicated or contrary to context, “circulating DNA” herein refers to cfDNA present in blood.

### Tumour Sequencing

Determining mutations present in a tumour sample may be performed by comparing DNA and/or RNA sequences isolated from tumour samples and comparative healthy samples from the same subject by exome sequencing, whole genome sequencing and/or targeted gene panel sequencing, for example.

Sequence alignment to identify nucleotide differences (e.g. single nucleotide variants or SNVs) in DNA and/or RNA from a tumour sample compared to DNA and/or RNA from a non-tumour sample may be performed using methods which are known in the art. The reference sample may be the germline DNA and/or RNA sequence.

The method of the present invention preferably uses multi-regional high-depth whole exome sequencing.

The method of the present invention relates to a subject specific method of defining a set of reagents to detect recurrence of a tumour in the subject.

In some embodiments of the invention, the whole genome and/or exome of the tumour is sequenced. In other embodiments of the invention, part of the genome and/or exome of the tumour is sequenced. Thus, all or part of the genome and/or exome of DNA from the tumour may be sequenced. The part of the genome and/or exome of the tumour which is sequenced may be for specific genes or chromosomal regions. For example, the part of the genome and/or exome of the tumour which is sequenced may include any of the genes or chromosomal regions defined in Table 1.

Whole or part of the genome and/or exome of the tumour may be sequenced and compared to whole or part of the genome and/or exome sequenced from healthy samples. A healthy sample may be a healthy tissue sample or a healthy blood sample taken from a part of the patient's body which does not have a tumour or is not affected by a tumour or tumour growth.

Whole or part of the genome and/or exome of the tumour or healthy sample may be sequenced to identify or define early clonal, clonal and/or subclonal mutations in the tumour. Determining the presence of early clonal, clonal and/or subclonal mutations in the tumour enables determination of the comparable early clonal, clonal and/or subclonal mutations in the cfDNA taken from the subject.

Whole or part of the genome and/or exome of the tumour or healthy sample may be sequenced to identify or define early clonal, clonal and/or subclonal mutations in the tumour. Determining the presence of early clonal, clonal and/or subclonal mutations in the tumour enables determination of the comparable early clonal, clonal and/or subclonal mutations in the cfDNA taken from the subject.

In some embodiments, whole or part of the genome and/or exome is sequenced from the whole tumour or from subsections of the tumour. It will be appreciated that a subsection of the tumour refers to a resected section of the whole tumour resected from the patient. Whole or part of the exome may be sequenced from one or more tumour subsections. Whole or part of the exome may be sequenced from at least one, at least two, at least three, at least four, at least five, at least six, at least seven, at least eight, at least nine, at least or up to ten subsections.

In some embodiments of the invention, the whole or part of the non-tumour genome and/or exome of the subject is sequenced. In some embodiments, the whole or part of the non-tumour genome and/or exome of the subject is sequenced and compared with the tumour genome and/or exome sequences to define the clonal and/or subclonal mutations found in the subject's tumour.

In some embodiments of the invention, sequencing all or part of the genome and/or exome of DNA from the tumour to define clonal and/or subclonal mutations in the tumour allows a set of reagents that will detect the presence of the clonal and/or subclonal mutation in cfDNA from the tumour to be defined.

### Definition and Detection of Clonal and/or Subclonal Mutations in cfDNA

A set of reagents is synthesised, defined and/or used to detect the presence of DNA from the tumour via the presence of early clonal, clonal and/or subclonal mutations. It will be appreciated that a set of reagents encompasses any reagents required to perform nucleotide sequence amplification and sequencing. It will also be appreciated that a set of reagents encompasses any reagents required to capture fragments of cfDNA. For example, a set of reagents may be a set of reagents for carrying out the CAPP-SEQ assay or a set of reagents for carrying out a digital PCR assay. In a preferred embodiment, the set of reagents is a set of reagents comprising probes and/or primers. Such probes and/or primers may be produced by known and routine techniques used in the art. Typically, the probes and/or primers will be for multiplex PCR. In a preferred embodiment, the set of reagents is a set of multiplex PCR probes and/or primers. The set of reagents for performing multiplex PCR is known to the person skilled in the art.

In some embodiments of the present invention, the cfDNA is obtained from the subject prior to removal of the tumour to identify mutations found in cfDNA from the tumour and using these mutations to define the set of reagents.

In some embodiments of the invention, the early clonal, clonal and/or subclonal mutations are defined based on which mutations occur in which tumour subsections. For example, early clonal, clonal and/or subclonal mutations may be defined on mutations occurring in the same tumour subsection or subsections and/or a different tumour subsection or subsections. In a preferred embodiment, early clonal, clonal and/or subclonal mutations in the tumour are defined from one or more tumour subsections. Early clonal, clonal and/or subclonal mutations in the tumour may be defined from at least one, at least two, at least three, at least four, at least five, at least six, at least seven, at least eight, at least nine, at least or up to ten subsections. In some embodiments of the invention, the set of detection reagents is capable of detecting one or more mutations. The set of detection reagents may be capable of detecting at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 mutations characteristic of each subsection of the tumour.

The method of the present invention relates to detecting recurrence of a tumour in a subject comprising analysing cfDNA with the subject specific set of reagents that will detect the presence of clonal and/or subclonal mutations from the tumour. The cfDNA is obtained from the subject subsequent or after the removal of the tumour and is analysed to determine whether or not the tumour has recurred by detection of the clonal and/or subclonal mutations from the tumour in the cfDNA. In a preferred embodiment, the analysis of the clonal and/or subclonal mutations in the cfDNA is a multiplex PCR.

In one embodiment, the method relates to detecting recurrence of a tumour in a subject by analysing cfDNA to identify early truncal mutations from the tumour. Since early truncal variants will be pervasive in the tumour and amplified due to genome doubling events, detection of these early events before genome doubling will provide greater sensitivity in terms of a biomarker predictive of disease activity and recurrence. A further advantage of detecting early mutations is that it is not necessary to sequence multiple regions, and so will reduce the time taken to carry out the method.

To increase the sensitivity of the method further, the early truncal mutations may be somatic SNVs in genes classified as being high-confidence early (pre-genome doubling) events or in chromosomal regions classified as being involved in high-confidence early (pre-genome doubling) amplification events. Examples of such genes and chromosomal regions for NSCLC are given in Table 1.

In another preferred embodiment, the set of regents synthesised, defined and/or used to detect the presence of DNA from the tumour via the presence of clonal and/or subclonal mutations is a set of multiplex PCR probes and/or primers that are used in the analysis of of the clonal and/or subclonal mutations in the cfDNA in a multiplex PCR reaction. A set of detection reagents may be a set of multiplex primers for use in a multiplex PCR reaction.

In some embodiments of the invention, the set of reagents detects one or more mutations characteristic of the trunk of the phylogenetic tree. The set of reagents may detect at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 mutations characteristic of the trunk of the phylogenetic tree.

In some embodiments of the invention, the set of reagents detects one or more mutations characteristic of the branch of the phylogenetic tree. The set of reagents may detect at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 mutations characteristic of each branch of the phylogenetic tree.

In some embodiments of the invention, the set of reagents detects one or more mutations characteristic of the trunk of the phylogenetic tree and one or more mutations characteristic of the branch of the phylogenetic tree. The set of reagents may detect at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 mutations characteristic of the trunk of the phylogenetic tree and at least one, at least two, at least three, at least four, at least five, at least six, at least seven, at least eight, at least nine, at least ten, at least fifteen, at least twenty, at least twenty-five, at least thirty, at least thirty five, at least forty, at least forty five, at least fifty, at least one hundred, at least one hundred and fifty, at least two hundred, at least two hundred and fifty, at least three hundred, at least three hundred and fifty, at least four hundred, at least four hundred and fifty, at least or up to 500 mutations characteristic of each branch of the phylogenetic tree.

In some embodiments, the cfDNA sample analysis is carried out at least 48 hours, from 48 hours to 7 days, from 7 to 14 days, from 14 days to one month, from one to two months, from two to six months, from six months to one year or over one year from the removal of the tumour.

In further embodiments, the cfDNA sample analysis is carried out on multiple occasions over a period of time, for example at regular intervals.

Detection of clonal and/or subclonal mutations characteristic of the tumour by analysing cfDNA obtained from the subject post tumour resection indicates recurrence of the tumour. The absence of clonal and/or subclonal mutations characteristic of the tumour by analysing cfDNA obtained from the subject post tumour resection indicates that the tumour has not recurred.

### Treatment Regimen based on Findings

The method of the present invention requires profiling a sample, for example cfDNA from a blood sample, for clonal and/or subclonal mutations characteristic of a tumour specific to a patient. Clonal and/or subclonal mutations are sensitive markers of tumour relapse. A treatment or treatments administered to the patient may be decisive based on the clonal variants, for example subclonal mutations in the “branches” of the phylogenetic tree. Detection of the subclonal mutations or subclones allows for the identification of the branches seeding relapse of the tumour. Identifying the branches seeding relapse may have implications in patient specific treatment options.

Based on the detection of the early clonal, clonal and/or subclonal mutations characteristic of the tumour in cfDNA, a personalised treatment regimen is created. Clonal and/or subclonal mutations characteristic for the recurring tumour present in cfDNA of a patient may be more susceptible to treatment by different methods. Based on the presence and/or rise of clonal and/or subclonal mutations characteristic for the tumour, a treatment regimen is designed. A treatment or treatments may include but are not limited to surgery. targeted small molecular therapy, immunotherapy, antibody therapy, adoptive T cell therapy, vaccine therapy, chemotherapy and/or radiotherapy. A treatment or treatments may be administered singly or in combination. A treatment or treatments may be adjuvant therapies, which may include, but are not limited to, adoptive T cell therapy, dendritic cell vaccination and cancer vaccination.

A treatment or treatments may comprise adjuvant chemotherapy, for example cisplatin and/or vinorelbine. A treatment or treatments may also comprise immunotherapy, for example with immune checkpoint inhibitors, adoptive immune T cell immunotherapy, cancer vaccination and/or CAR-T cells. A treatment or treatments may also comprise T cell therapy against clonal neo-antigens or modulating adoptive T cell therapy in real time by tracking clonal variants that encode patient-specific neoantigens, i.e. tumour mutations that make the protein visible to the host immune system. A treatment or treatments may comprise using targeted therapy, for example Afatanib, against a targetable alteration such as an EGFR mutant or ERBB2 amplification.

The presence and/or rise of early clonal and/or clonal mutations in cfDNA may be used to monitor response to treatment. The presence and/or rise of early clonal and/or clonal mutations in cfDNA may be used to monitor response to adjuvant treatment, for example adjuvant chemotherapy. The detection of early clonal, clonal and/or subclonal clonal mutations in cfDNA indicates lack or response to treatment. The absence of early clonal, clonal and/or subclonal mutations in cfDNA correlates with successful treatment outcome.

The presence and/or rise of early clonal, clonal and/or subclonal mutations in cfDNA characteristic of a tumour in a specific patient during treatment may change the treatment regimen. The presence and/or rise of early clonal, clonal and/or subclonal mutations in cfDNA characteristic of a tumour in a specific patient during adjuvant treatment, for example adjuvant chemotherapy, may change the treatment regimen. Such a presence and/or rise of early clonal, clonal and/or subclonal mutations in cfDNA characteristic of a tumour in a specific patient during treatment and/or adjuvant treatment, for example adjuvant chemotherapy, may be used as a tool to stop treatment, use an alternative treatment, escalate treatment, for example to a clinical trial, and/or de-escalate treatment. For example, the treatment or adjuvant treatment may not be treating the tumour in the specific patient, or the treatment or adjuvant treatment may be toxic to the specific patient. The rationale behind stopping treatment, using an alternative treatment, escalating treatment and/or de-escalating treatment may be based on the presence, absence, rise and/or fall of early clonal, clonal and/or subclonal mutations in cfDNA.

The presence, absence, rise and/or fall of early clonal, clonal and/or subclonal mutations in cfDNA may facilitate the clinician in deciding what treatment to administer to the specific patient. Based on the the presence, absence, rise and/or fall of early clonal, clonal and/or subclonal mutations in cfDNA, the clinical may decide to administer or not to administer adjuvant chemotherapy. The decision to administer or not to administer adjuvant chemotherapy based on the presence, absence, rise and/or fall of early clonal, clonal and/or subclonal mutations in cfDNA may also be based on the levels of early clonal, clonal and/or subclonal mutations in cfDNA at a time period post-surgery. The cfDNA sample analysis may be carried out on multiple occasions over a period of time post-surgery, for example at regular intervals. The cfDNA sample analysis may be carried out at least 48 hours, from 48 hours to 7 days, from 7 to 14 days, from 14 days to one month, from one to two months, from two to six months, from six months to one year or over one year post-surgery. The cfDNA sample analysis may be carried out between 4 to 6 weeks post-surgery.

The invention will now be further described by way of Examples, which are meant to serve to assist one of ordinary skill in the art in carrying out the invention an are not intended in any way to limit the scope of the invention.

### EXAMPLES

### Materials and Methods

### Introduction

A study demonstrated the successful detection of cancer-relevant point mutations in the plasma of cancer patients.

The mutation profile of 4 lung cancer tumours was determined by whole exome sequencing (WES) or AmpliSeq, and a subset of those mutations were successfully detected in the corresponding plasma samples using a multiplex PCR-Next-Generation Sequencing (mPCR-NGS) method.

The mPCR-NGS method was used to detect and track over time cancer-specific mutations in the plasma of cancer patients, and to evaluate the utility of the method in monitoring disease progression through treatment. The first phase of the project was the determination of the baseline mutation profile in the plasma of 50 treatment-naïve lung cancer patients.

Purified genomic DNA samples from several tumour regions (1-7 regions per tumour), purified germline DNA samples, and intact plasma samples from 50 patients were provided. The mutation profile of all of the tumour regions was previously determined by whole exome sequencing (WES) and AmpliSeq, and a subset of mutations per patient was determined. Those mutations include both driver and passenger mutations, and both clonal and sub-clonal mutations. The full mutation profile of each tumour was used to reconstruct the phylogenetic tree of each tumour.

Based on these data, multiplex PCR assays were designed, corresponding primers were ordered, primer pools were prepared, primer pools quality controlled and the mPCR protocol optimised for each pool. Plasma cfDNA was purified, quantified and converted into libraries. The libraries were then used as input into the mPCR, the products were sequenced and analysed. A similar protocol was applied to the genomic DNA from tumour and matched normal samples.

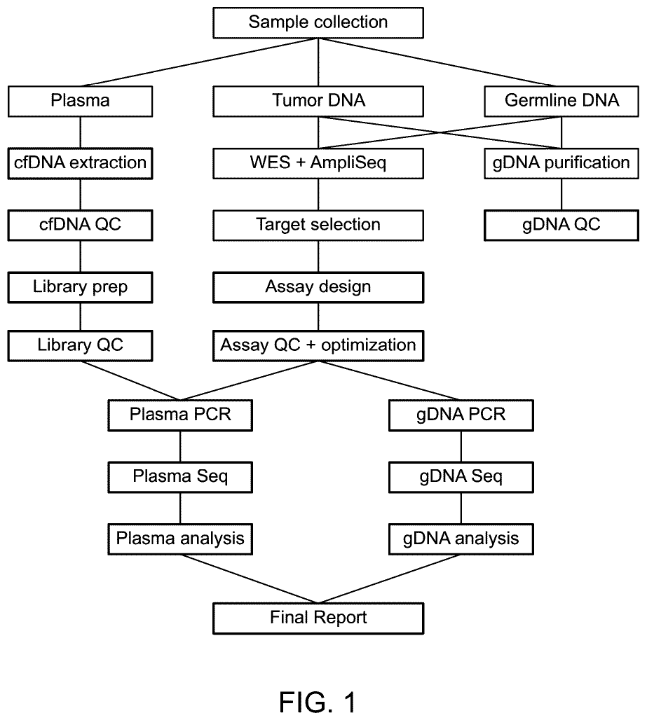

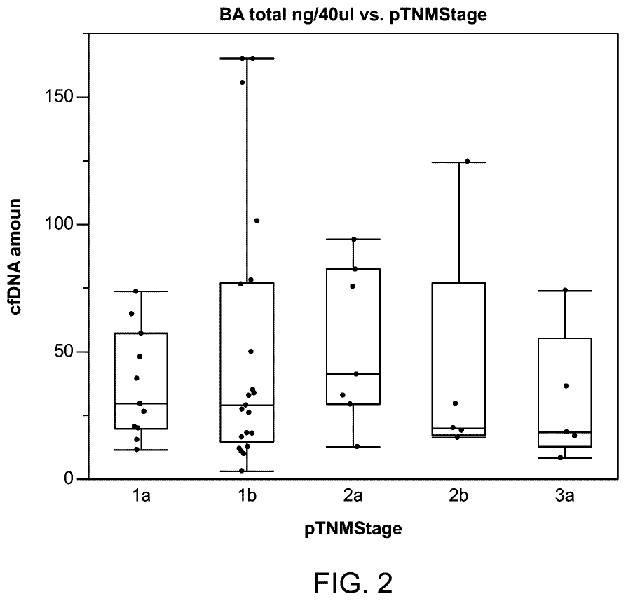

A workflow diagram is shown in FIG. 1. The total number of SNVs per sample and the working assays sorted by driver category are shown in FIG. 2.

### Study Population

For each of the first 50 patients, 4-5 mL of plasma obtained before tumour resection and prior to any therapy was isolated. Plasma samples were aliquoted in 2 mL tubes.

Purified genomic DNA from up to 7 tumour subsections, from affected lymph nodes (where available), and from the white blood cell fraction (referred to as the matched normal) were purified and 500 ng purified DNA from each sample, normalized at 10 ng/uL, was obtained.

The mutation profile, which included single nucleotide variants (SNVs) for each tumour subsection, was determined using whole exome sequencing. The full mutation profile of each tumour was used to reconstruct the phylogenetic tree of each tumour.

PyClone was used to identify clusters of SNVs, and calculate their cancer-cell fraction. This was used to categorize SNVs as either clonal or subclonal. The driver category of each SNV was determined and provided as the driver category (1-4, where 1 is most likely to be a driver mutation, and 4 is the least likely). For each patient, up to 108 SNVs, spanning all driver categories and including clonal and subclonal mutations, were identified. The detected allele fractions of each SNV in each tumour subsection, lymph node and matched normal DNA sample along with PyClone clonal/subclonal cluster information were also identified.

For each patient, the following information was available: tumour size (mm), tumour location (lung lobe), tumour stage, tumour histological type, number of lymph nodes affected, vascular invasion status, as well as de-identified information on the collecting hospital.

### Assay Design and Protocol Optimization

The standard assay design pipeline was used to design Right and Left PCR primers for all given SNVs. We refer to a pair of Right and Left PCR primers targeting a SNV as an assay for that particular SNV. Note that it is possible for one assay to cover more than 1 target SNV, if they were in close proximity. For every pair of assays, the probability of forming primer-dimer was calculated using the standard pipeline. The SNV allele-fraction data in each tumour was used to reconstruct phylogenetic trees using LICHeE (Lineage Inference for Cancer Heterogeneity and Evolution). The list of assays for each sample were filtered to remove primers that were predicted to form primer dimers while giving strong priority to assays covering driver 1 and 2 SNVs. The remaining assays were used to create 5 balanced pools, each containing assays of 10 patients. All assays pooled together were compatible, meaning there were no primers predicted to form primer-dimer in a pool. At each step, the assays were chosen such that:
Assays covering driver 1 and 2 SNVs have the highest priority
For each patient number of selected SNVs per branch is proportional to the total number SNVs of that branch from the reconstructed phylogenetic tree. More specifically, we tried to have a uniform sampling of SNVs from branches in the reconstructed phylogeny tree, making sure selected assays give provide a good coverage of the reconstructed tree.
   The final design consisted of 972 assays, equally distributed among the 5 pools, and containing 15-20 assays for each sample.

### Pool Quality Control (QC) and Optimization

The 972 primer pairs were synthesized by Integrated DNA Technologies (IDT) in individual wells, desalted, and normalized to 100 uM. The assays were pooled according to the pooling scheme, and each pool was used in a combined QC/optimization experiment. For the optimization experiment, several PCR parameters were varied and the effects on the sequencing performance, as well as the number of failed assays, were evaluated from the sequence data. The PCR conditions that yield the best percentage of on target reads, depth of read uniformity, and error rate were determined. Primers that were responsible for the majority of primer dimers were identified and removed from each pool (for each primer removed, its corresponding partner was also removed). Following this step, 908 total assays remained, equally distributed among the 5 pools.

### DNA Extraction and QC

All the plasma aliquots from each patient were pooled prior to cfDNA extraction, and the hemolysis grade of each pooled plasma sample was evaluated visually (no hemolysis, mild hemolysis, or severe hemolysis). cfDNA was extracted using the Qiagen NA kit following a protocol optimized for 5 mL of plasma. All cfDNA samples were quality controlled on Bioanalyzer High Sensitivity chips. The same Bioanalyzer High Sensitivity runs were also used to quantify the cfDNA samples by interpolation of the mononucleosomal peak height on a calibration curve prepared from a pure cfDNA sample that was previously quantified. This was necessary because cfDNA sometimes contains an intact DNA fraction that overlaps with the high size marker on the chip, which makes quantification of the mononucleosomal peak unreliable. A representative subset of the purified genomic DNA samples (from tumour subsections, lymph nodes and white blood cells) were quantified using the QuantIt DNA High Sensitivity kit. All of the samples quantified were in the expected range (˜10 ng/uL).

cfDNA Library Preparation

The entire cfDNA amount from each plasma sample was used as input into Library Prep using the library prep kit and following the kit instructions. For two samples with extremely high cfDNA amounts, the input amount into Library Prep was restricted to ˜50,000 genome equivalents (165 ng). The libraries were amplified to plateau and then purified using Ampure beads following the manufacturer's protocol. The purified libraries were quality controlled on the LabChip.

cfDNA Multiplex PCR and Sequencing

The library material from each plasma sample was used as input into multiplex PCR (mPCR) using the relevant assay pool and an optimized plasma mPCR protocol. The mPCR products were barcoded in a separate PCR step, and the barcoded PCR products were pooled according to the assay pooling information into 5 pools. The pools were purified using Ampure beads following the manufacturer's protocol, quality controlled and quantified on a Bioanalyzer DNA1000 chip. Each pool contained 10 cancer plasma libraries and 20 negative controls (prepared from cfDNA extracted from presumed healthy volunteers). All 20 control specimens were obtained by the clinical research department under IRB-approved protocols with proper patient consent following Good Clinical Practice (GCP). Samples were obtained from one of three sources: healthy volunteers, Advanced Biomedical Research, Inc., or StemExpress. Each pool was sequenced on a separate HiSeq2500 Rapid run with 50 cycle paired end single index reads.

gDNA Multiplex PCR and Sequencing

The genomic DNA samples were used as input into a similar mPCR using the relevant assay pools and an optimized genomic mPCR protocol. The mPCR products were barcoded in a separate PCR step, and all the barcoded products were combined into one pool. The pool was purified using Ampure beads following the manufacturer's protocol, QCed and quantified on a Bioanalyzer DNA1000 chip. The pool was sequenced on a single HiSeq2500 Rapid run with 50 cycle single end single index reads.

### Subclonal Deconstruction

To determine whether mutations were clonal or subclonal, and the clonal structure of each tumor, a modified version of PyClone was used. For each mutation, two values were calculated, obsCCF and phyloCCF. obsCCF corresponds to the observed cancer cell fraction (CCF) of each mutation. Conversely, phyloCCF corresponds to the phylogenetic CCF of a mutation. To clarify the difference between these two values, consider a mutation present in every cancer cell within a tumor. A subclonal copy number event in one tumor region may lead to loss of this mutation in a subset of cancer cells. While, the obsCCF of this mutation is therefore below 1, from a phylogenetic perspective the mutation can be considered clonal as it occurred on the trunk of the tumor's phylogenetic tree, and, as such, the phyloCCF may be 1.

To calculate the obsCCF of each mutation, local copy number (obtained from ASCAT), tumor purity (also obtained from ASCAT), and variant allele frequency were integrated. In brief, for a given mutation we first calculated the observed mutation copy number, nmut, describing the fraction of tumor cells carrying a given mutation multiplied by the number of chromosomal copies at that locus using the following formula:

$${n}_{mut} = VAF \frac{1}{p} [ {PCN}_{t} + {CN}_{n} ( 1 - p ) ]$$

where VAF corresponds to the variant allele frequency at the mutated base, and p, CNₜ, CNₙ are respectively the tumor purity, the tumor locus specific copy number, and the normal locus specific copy number (CNₙ was assumed to be 2 for autosomal chromosomes). The expected mutation copy number, n_{chr}, using the VAF was calculated and a mutation to one of the possible local copy numbers states using maximum likelihood was assigned. In this case only the integer copy numbers were considered.

All mutations were then clustered using the PyClone Dirichlet process clustering. For each mutation, the observed var_count was used and ref_count was set such that the VAF was equal to half the pre-clustering CCF. Given that copy number and purity had already been corrected, the major allele copy numbers to 2 and minor allele copy numbers to 0 and purity to 0.5 were set: allowing clustering to simply group clonal and subclonal mutations based on their pre-clustering CCF estimates. We ran PyClone with 10,000 iterations and a burn-in of 1000, and default parameters, with the exception of —var_prior set to ‘BB’ and —ref_prior set to ‘normal’.

To determine the phyloCCF of each mutation, a similar procedure to that described above was implemented, with the exception that mutations were corrected for subclonal copy number events. Specifically, if the observed variant allele frequency was significantly different from that expected (P<0.01, using prop.test in R) given a clonal mutation, we determined whether a subclonal copy number event could result in a non-significant (P>0.01) difference between observed and expected VAFs. The pre-clustering CCF for each mutation was then calculated by divided nₘᵤₜ by n_{chr}. Subclonal copy number events were estimated using the raw values from ASCAT output.

To ensure potentially unreliable VAFs of indels did not lead to separate mutation clusters, each estimated indel CCF was multiplied by a region specific correction factor. Assuming the majority of ubiquitous mutations, present in all regions, are clonal, the region specific correction factor was calculated by dividing the median mutation CCF of ubiquitous mutations by the median indel CCF of ubiquitous indels.

### Timing of Mutations

Individual mutations (SNVs, DNVs and indels) were timed as clonal or subclonal through PyClone clustering as described above. For clonal timing, the values from phyloCCF were used. Clonal mutations were then further timed as early, late or untimed clonal. This was performed first within each tumor region relative to overlapping copy number events as previously described. In brief, mutations in parts of the genome with at least two copies of the major allele were preliminarily classified as early if the mutation copy number (described above) was >1, and late if it was <=1. Temporal analysis was then performed across all regions. Mutations classified as clonal were called as “clonal early” if a given mutation was early across all regions, or, if the mutation was early in the majority of regions. A clonal mutation was called as “clonal late” if it was called as late across all regions, or if it was late across the majority of regions. Any clonal mutations that could not be timed as either early or late, was classified as “clonal untimed”.

### Timing of Chromosomal Arm SCNA

Whole chromosomal arm gain and loss were called as clonal and subclonal as described above. Clonal chromosomal arm gain was further timed by the mutation copy number of all mutations on a given arm. All mutations mapping to the arm gain was identified, and the across-region average ratio of mutations with a mutation copy number within one copy of the major allele was determined. If the majority of mutations showed a copy number within one copy of the major allele, indicating that the mutations occurred prior to increase in copy number and were gained with the chromosomal arm, the chromosomal arm gain was called as “clonal late”. If the majority of mutations showed a lower copy number, indicating that the mutations occurred after chromosomal arm gain, the copy number gain was called as “clonal early.” If there were not enough mutations mapping to regions of chromosomal arm gain, the chromosomal arm gain was called as “clonal untimed”. For clonal chromosomal arm loss, we timed the losses relative to LOH status in genome doubled tumors. Any chromosomal arm loss showing LOH in a genome doubled context is likely to have occurred prior to genome doubling as it would otherwise require two independent hits. Thus, chromosomal arm loss in a genome doubled context showing LOH were called as “clonal early”, chromosomal arm loss in a genome doubled context without LOH were called as “clonal late”, and chromosomal arm loss not in a genome doubled context were called as “clonal untimed”.

### Phylogenetic Tree Construction

Mutations were clustered across tumour regions as described above. Mutation clusters with at least 5 mutations assigned were included in the phylogenetic analysis. Only tumors with at least two regions with good copy number data and at least two mutation clusters were included in the analysis (292 tumor regions from 90 tumors). To infer phylogenetic relationship between clusters of mutations, a one sided t-test was performed between the phyloCCF values of each mutation cluster pair, in all tumor regions. A p-value threshold of 0.025 was applied to determine if a given cluster of mutations was significantly smaller than another cluster. If cluster A was significantly smaller than B in at least one region, and cluster B is not significantly smaller than cluster A in any regions, then cluster A was determined to have descended from cluster B. This would occasionally result in impossible phylogenetic relationships, where e.g. a cluster A was annotated as descended from two different clusters, B and C, both descended from D, creating a circle in the phylogenetic tree. To resolve this, all circles were identified, and resolved by sequentially removing the cluster containing the fewest mutations until no circles would be found in the phylogenetic tree structure. All phylogenetic trees were manually verified, by comparing the tree structure with region-by-region scatter plots of pyclone-clustered CCF values. Smaller mutation clusters removed for causing circles were attempted put back by comparing the CCF values across regions both before and after correcting for copy number changes. Occasionally in particular smaller mutation clusters had large confident intervals in specific regions, potentially due to copy number changes, causing erroneous placement in the phylogenetic tree structure. This was resolved in the manual verification step.

### Mutational Signature Analysis

Mutational signatures were estimated using the deconstructSigs package in R. Signature 1A, 2, 4, 5, 13 were considered. For temporally dissected mutational signatures, mutational signature analysis was only applied if at least 15 mutations were present. Similarly, subclone specific mutational signature analysis was restricted to subclones with at least 15 mutations.

### Data Analysis

### Plasma SNV Calls

Each plasma sequencing run contained 10 cancer samples and 20 negative control samples, all sequenced with the corresponding mPCR pool. The set of SNVs covered by assays in a pool were considered as target SNVs for the associated run. At each SNV position, an error model was built using all of the 20 negative control samples plus the 9 cancer samples that were not expected to contain that particular SNV. Samples with high plasma VAF (>20%) among the putative negatives were considered to have possible germline mutation and were excluded from the background error model3. A confidence score was calculated for each target SNV based on the background error model. A positive plasma SNV call was made if the calculated confidence for that mutation in the corresponding plasma sample passed our confidence threshold.

The same calling strategy with the same confidence threshold was used to attempt to make SNV calls on the remaining samples (including other cancer samples and control samples) that were not expected to contain the given SNVs. The SNVs that were detected in this way were considered ‘false positive’. The false positive rate was 0.24% (at the mutation level).

### Tissue SNV Calls

All the tumour subsections, lymph node samples and matched normal samples were sequenced on one sequencing run. For each sample (tissue subsection, lymph node or matched normal), only the appropriate mPCR pool was used. The tissue VAF for each expected mutation was determined directly from the sequence data as (mutant DOR)/(total DOR). Only mutations with tissue VAF >5% were considered positive.

### SNV Assay Pool Creation and Validation

cfDNA Extraction

Up to 5 ml of plasma per case was available for this study. The entire volume of plasma was used for cfDNA extraction. cfDNA was extracted using the QIAamp Circulating Nucleic Acid kit (Qiagen) and eluted into 50 ul DNA Suspension Buffer (Sigma). The purified cfDNA was stored frozen at −20 oC until use.

cfDNA QC and Quantification

Every cfDNA sample was QCed and quantified on the Bioanalyzer High Sensitivity (Agilent). Plasma cfDNA consists of a main mono-nucleosomal peak (˜160 bp): for some samples, di-nucleosomal and tri-nucleosomal peaks are visible (at ˜320 bp and ˜500 bp, respectively). The library prep method used selectively amplifies the mono-nucleosomal fraction of cfDNA. Because a high molecular weight DNA fraction sometimes overlaps the upper marker used on the Bioanalyzer, quantification is not always reliable. Consequently, the amount of cfDNA analyzed was estimated from the peak height of the mono-nucleosomal peak using a standard curve.

### SNV Assay Design

Natera's standard assay design pipeline was used to design forward and reverse PCR primers for all SNVs detected in tumour samples: each pair targeting an SNV comprises the assay for that SNV. If two SNVs were in close proximity, a single assay could cover more than 1 target SNV. For some SNVs, it was not possible to design an assay. When creating assay pools, the probability of every pairing of two assays to form primer-dimers was calculated. For each patient sample, an optimal list of assays with no predicted primer-dimer was selected, while giving strong priority to assays covering driver SNVs. These remaining assays were used to create 10 balanced pools, each containing the assays for 10 patients' SNVs and on average 20 assays per each patient. Assays were always chosen such that, 1) assays covering driver SNVs had highest priority, and 2) there was uniform sampling of SNVs from branches in the reconstructed phylogeny tree. The final design for baseline cohort consisted of 1835 assays, equally distributed among the 10 pools, and contained 15-20 assays for each sample.

For the longitudinal cohort, up to 10 extra assays were added for adenocarcinoma samples. Designed assays were used to create 5 balanced pools, each containing 5 patients. In total 598 assays were analyzed for the longitudinal cohort.

### SNV Pool QC

The SNV assays, each consisting of a forward and reverse primer, were ordered from IDT (Coralville, IA) as individual oligos in 96-well plates, desalted and normalized to 100 uM each. The oligos were pooled according to the pooling scheme and each pool was QCed by running the multiplex PCR and sequencing protocol using one plasma cfDNA library from a healthy subject. For each pool, the sequencing data was analyzed to determine the amount of primer-dimer reads and to identify drop-out assays. If a large proportion of sequencing reads result from primer-dimers, the primer-dimer reads were analyzed to determine the primers contributing to dimer formation, and primers that contributed to most primer-dimer reads were removed. A final pool consisting of only the functional assays from each pool was created and used for SNV detection in the corresponding plasma samples.

### Analytical Validation

Twenty synthetic spikes were mixed at equimolar ratios and used to prepare a library. This library was titrated into a library prepared from mononucleosomal DNA (10,000 copies) from a normal cell line (AG16778 from Coriell, Camden, NJ). The synthetic spikes matched 20 SNVs grouped into Pool 1 of the baseline samples. Each synthetic spike consisted of a 160 bp dsDNA oligo having the SNV at position 80, flanked by the normal sequence at that position in the human genome.

The library of 20 synthetic spikes was titrated into the mononucleosomal DNA library at 2.5%, 0.5%, 0.25%, 0.1%, 0.05% (each in triplicate), and 0.01%, 0.005% and 0.001% (each in quadruplicate): the mononucleosomal library was also run without spikes, in triplicate, as a negative control. Because preparing spiked samples at such low levels is either subject to sampling noise (0.01% spikes into 10,000 genomic copies background is equivalent to one mutant copy) or is not possible (at levels less than 0.01%), samples were mixed as libraries. Furthermore, because the library prep process has an efficiency of ˜80%, for low copy mutant samples it would not be possible to know the number of mutant molecules represented in the final library. Library mixing allows the testing of the SNV-detection step without interference from the library-prep step.

Following library mixing and sequencing, the 20 spiked-in SNVs were analyzed. The measured VAF of each spike for the samples with 2.5% nominal input was used to calculate an input correction factor (measured VAF/2.5%); that was applied to the other inputs of the corresponding spike. The measured VAF differed from the nominal input (for example, 2.5%) most likely because the mononucleosomal fragmentation pattern is not entirely random (so the actual number of DNA fragments that can be detected with a specific PCR assay differs from the average input of 10,000 copies). Because of this, the actual input levels differ from the nominal inputs, and the sensitivity is measured for input intervals. The input intervals were chosen such that there are a meaningful number of samples in each interval.

### Plasma SNV Analysis

Forty microliters of the extracted cfDNA from each case was used as input into library preparation using the Natera Library Prep Kit, which included blunt-end repair, A-tailing, universal adaptor ligation, PCR amplification to plateau and purification of the amplified material. All purified libraries were QCed on the LabChip GX 5k DNA chip. Successful libraries had a single peak at ˜250 bp.

The amplified libraries were then analyzed by mPCR-NGS. PCR primers targeting SNVs detected in the analysis of the tumor samples (up to 20 targets/sample). Optimal PCR conditions for each pool were as described in Jamal Hanjani et al. Ann Oncol. 2016.

Each PCR assay pool was used to amplify the SNV targets from the 10 corresponding samples and 20 negative control samples (plasma libraries prepared from healthy subjects). Each amplicon pool was sequenced on one Illumina HiSeq 2500 Rapid Run with 50 cycles paired-end reads using the Illumina Paired End v1 kit with an average target DOR of ˜40,000 per assay.

### Statistical Methods

Each plasma sequencing run contained 10 cancer samples and 20 negative control samples, all sequenced with the corresponding mPCR pool. The set of SNVs covered by assays in a pool were considered as target SNVs for the associated run. Target assays with <1000 reads in the plasma samples were considered failures and were not analyzed further. At each SNV position, an error model was built using all of the 20 negative control samples plus the 9 cancer samples that were not expected to contain that particular SNV (based on tumor-tissue sequencing). Samples with high plasma VAF (>20%) among the putative negatives were considered to have possible germline mutation and were excluded from the background error model. A confidence score was calculated for each target SNV based on the background error model. A positive plasma SNV call was made if the calculated confidence for that mutation in the corresponding plasma sample passed our confidence threshold.

The same calling strategy with the same confidence threshold was used to attempt to make SNV calls on the remaining samples (including other cancer samples and control samples) that were not expected to contain the given SNVs. The SNVs that were detected in this way were considered ‘false positive’. The false positive rate was 0.24% (at the mutation level).

### Tumor Tissue and Germline DNA SNV Analysis

Purified gDNA from tumor subsections and germline DNA samples (extracted by CRUK and normalized to 10 ng/ul) were used to confirm the target SNVs in each patient. For this, the gDNA (70 ng/rxn) was used as input into mPCR using the corresponding primer pools and the same PCR conditions that were used on the plasma libraries. The samples were sequenced on one Illumina HiSeq 2500 Rapid Run with 50 cycles single-end reads using the Illumina Single End v1 kit with an average target DOR of ˜4,000 per assay. Sequence data was used to determine the variant allele frequency (VAF) of each of the expected SNV in each sample. The VAF determined in this way was compared to the VAF determined by CRUK.

### Example 1—cfDNA Extraction and Analysis

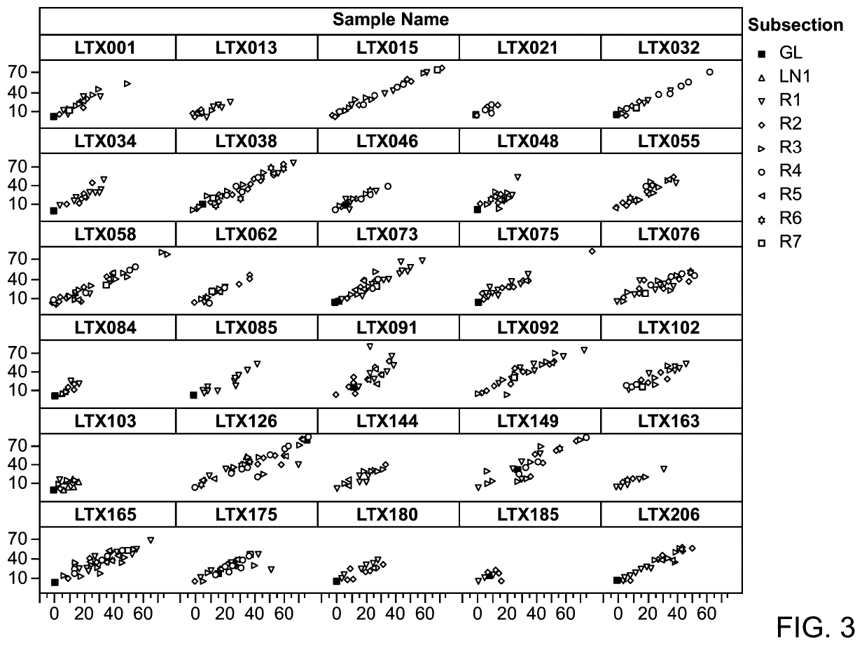

The distribution of cfDNA concentrations for the 50 plasma samples (FIG. 3) follows the expected distribution based on 5 mL of plasma (median of 2,200 genome copy equivalents per mL of plasma). The cfDNA concentrations, the hemolysis grade (visually estimated) and the qualitative evaluation of the cfDNA size profile (visually estimated from the Bioanalyzer traces) are shown in Table 3.

### Example 2—Variant Allele Frequency (VAF) Analysis in Tumour Subsections

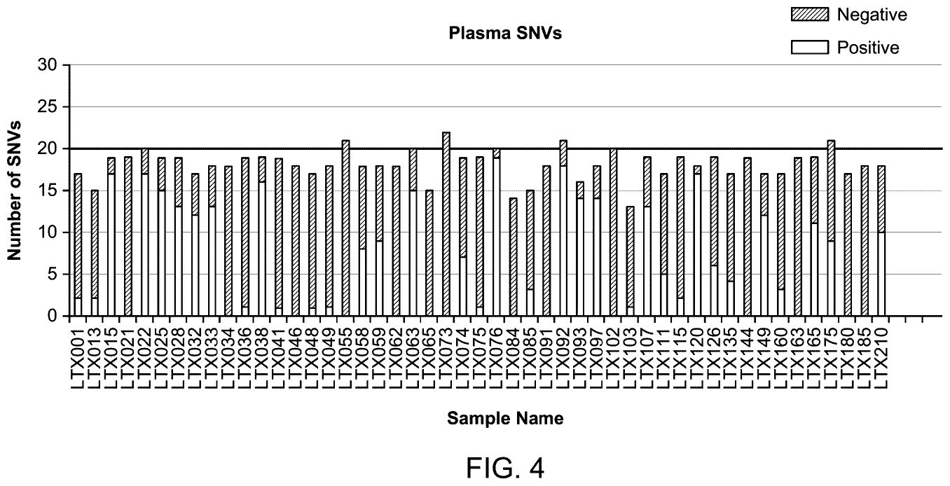

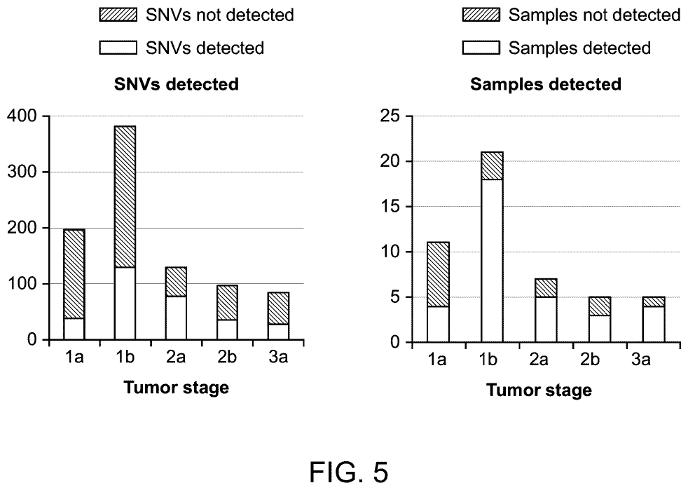

The sequence data from each of the tumour subsections was analysed to determine the variant allele frequency of each SNV in each tumour subsection, lymph node and matched normal sample. This data was compared with the similar data. For most samples, the tissue VAF values from each tumour subsection closely matched the tissue VAF values determined (FIG. 4). However, there were a large number of samples in which significant discrepancies were seen (FIG. 5). Three types of discrepancies are observed:
(i) For one or two subsections, all the VAFs were 0 or close to 0 in the analysis, but were non-zero (and span the range of VAFs seen in other subsections of the same sample) in the subsequent analysis (e.g.: LTX041, LTX111);
(ii) For several assays, the VAFs were 0 in analysis, but were non-zero (and span the range of VAFs seen in other subsections of the same sample) in the subsequent analysis, but no clustering by subsection was seen with this discrepancy mode (e.g.: LTX093, LTX074);
(iii) For several assays or regions, none of the assays failed but concordance between VAFs obtained in the two analyses was generally poor (e.g.: LTX063, LTX059).

### Example 3—Plasma SNV Detection

One sample (LTX206, with 19 assays) failed sequencing and was removed from the analysis (this sample will be rescued in a subsequent experiment). 889 assays covering 911 SNVs remained. Assays with a depth of read of less than 1,000 were considered failed, and their corresponding SNVs were marked as “no call”. In total 21 “no call” SNVs were removed from the analysis; 890 total SNVs were analysed.

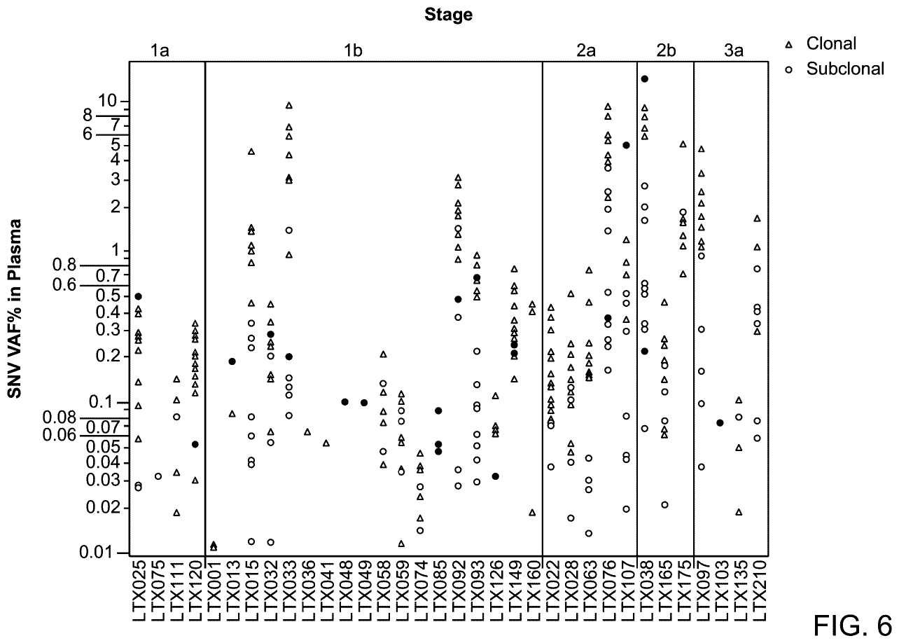

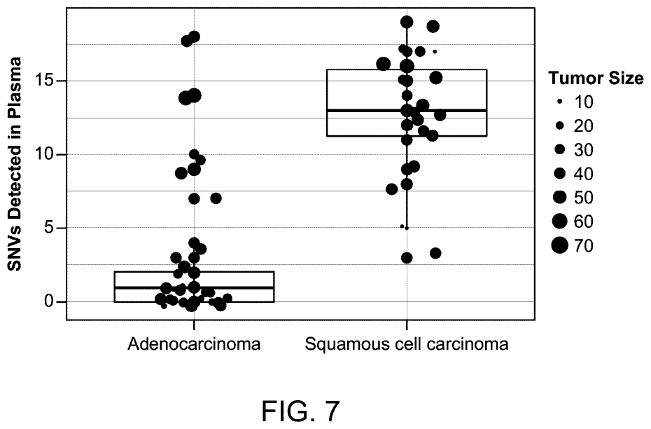

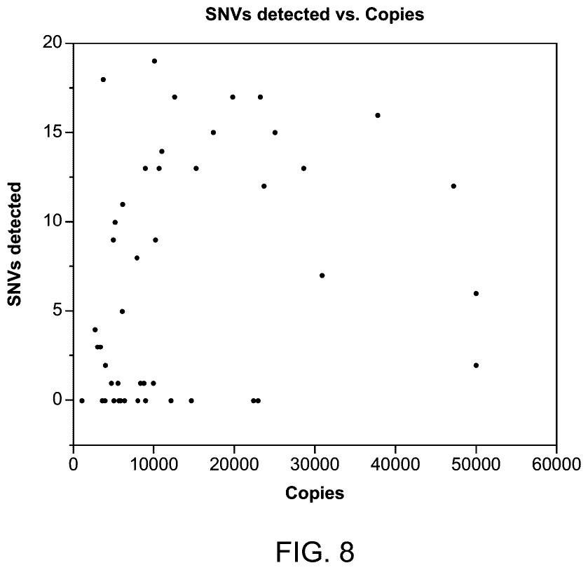

The overall SNV detection rate in plasma was 35.5% (310/890), which is similar to what we observed o the pilot study. The SNVs that were not detected were probably due to the fact that the mutations were not present in the plasma sample. The average mutant allele frequency for the SNVs detected with high confidence was 0.875% (range, 0.011-13.93%). A cancer sample was considered as ‘detected in plasma’ if ≥1 SNV expected to be present in that sample was confidently detected in plasma. Using this definition, the overall sample detection rate in plasma was 69% (34/49 samples), and for those, and the average number of SNVs detected in plasma was 9.1 (range, 1-19). See FIG. 6 for a visual representation of the SNVs detected in plasma. A breakdown by SNV and by sample is shown in FIG. 7. The number of SNVs detected in plasma for each sample is shown in Table 4, and the full data for all the SNVs analysed is shown in Table 5. The plasma VAF for the SNVs that were detected from each sample as a function of SNV clonal status is shown in FIG. 8.

### Example 4—Additional Findings

In addition to target SNVs, we examined every position amplified by our assays both in tumour and plasma. In total, 16 somatic novel SNVs not reported were identified from tissue samples. Among those, 7 were called with their corresponding plasma samples as well. As we used very stringent confidence thresholds to avoid potential false positives, we are highly confident about the detected mutations. They are shown in Table 6.

### Example 5—Factors Influencing SNV Detection in Plasma

Several factors might influence plasma SNV detection. Here, we aimed to determine the factors with the most significant association. Factors analysed for association included: tumour size (Lesion 1 SizeDer values in Table 5), stage, tumour histological type, lymph node status, vascular invasion, location by lobe of the lung, the SNV frequencies in tumour subsections, clonal information for each SNV and the amount of cfDNA. The multivariate analyses for both sample-level and mutation-level plasma SNV detection are described below. Sample-level analysis aimed to identify factors affecting the number of detected SNVs in a plasma sample. Mutation-level analysis aimed to identify factors affecting the call for a plasma SNV.

### Sample-Level Analyses

Poisson regression analysis, adjusted for over dispersion, indicated a highly statistically significant association between the number of SNVs detected in plasma and the histological type. Estimated model coefficient corresponding to histological type of 1.29 with standard error 0.30 (p-value=2.48e-05). This association did not change when adjusting for tumour size, stage, or site.

We also found a weak association between number of SNVs detected in plasma and tumour size within the same Poisson regression model (estimated model coefficient for tumour size was 0.02 with a standard error of 0.01, p-value=0.0682). Tumour size and stage were correlated, and seem to provide similar information about the number of SNV detectable in plasma. Once tumour size was taken into account, tumour stage appeared to add no additional information that would explain the variation in the number of SNVs detected in plasma (model coefficient for tumour stage was not statistically different from zero at the standard significance level of 0.05, p-value=0.3677).

We interpret the model coefficients as follows. Compared with adenocarcinoma, squamous-cell tumours with the same tumour size, stage, and site have on average exp(1.29) ˜ 3.7 times more SNVs detected in plasma. For fixed values of histological subtype, tumour stage and tumour site, an increase in tumour size by one unit (mm) results in a multiplicative increase in SNV count of exp(0.02)=1.02, (an approximate 2% average increase).

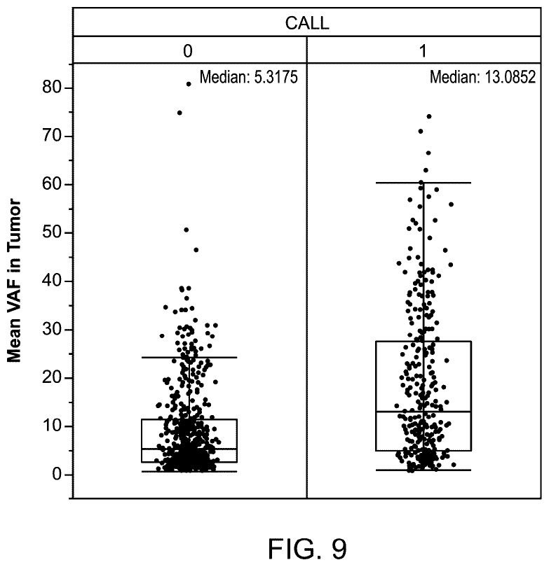

In summary, the most important predictor of whether a particular tumour was detected in the plasma appeared to be histological type: 100% of the squamous cell carcinomas (SQCC) were detected in plasma, whereas only 52% (15/29) of the adenocarcinomas (ADC) were detected (FIG. 9).

There were only one carcinosarcoma tumour and one adenosquamous tumour in this cohort, so no conclusions about their general detectability in plasma could be derived about those tumour types at this time.

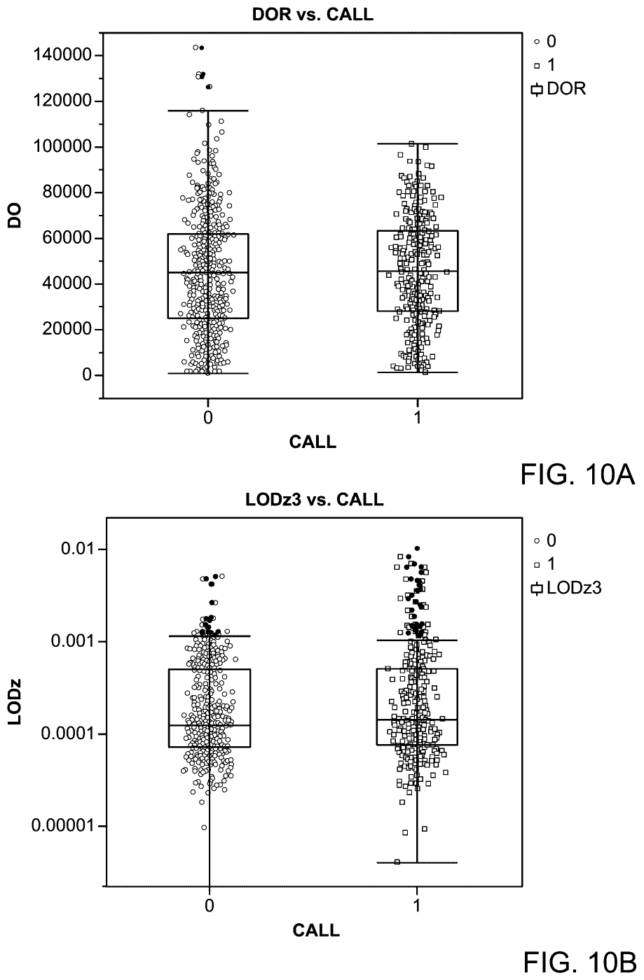

There was no apparent correlation between the amount of cfDNA and the number and proportion of SNVs detected in the plasma samples. However, all samples with high input (>25,000 copies) had ≥1 SNV detected in plasma (FIG. 10).

### Mutation-Level Analyses

A regression analysis was performed to determine the variables that can be used to predict detection of mutations in plasma. More specifically, a binary response variable was used to annotate the mutations as ‘called’ or ‘not called’. Mean VAF in tumour refers to the average mutation VAF among all tumour regions. Clonal/subclonal information was determined using PyClone. Logistic regression analysis showed that the following variables had statistically significant association with the detection of a mutation (with p-values <5%):
Histological type (p=8.3e-30)
Mean VAF in Tumour (p=4.3e-6)
Clonal status (p=1.6e-4)
Size of the tumour (p=3.5e-4)

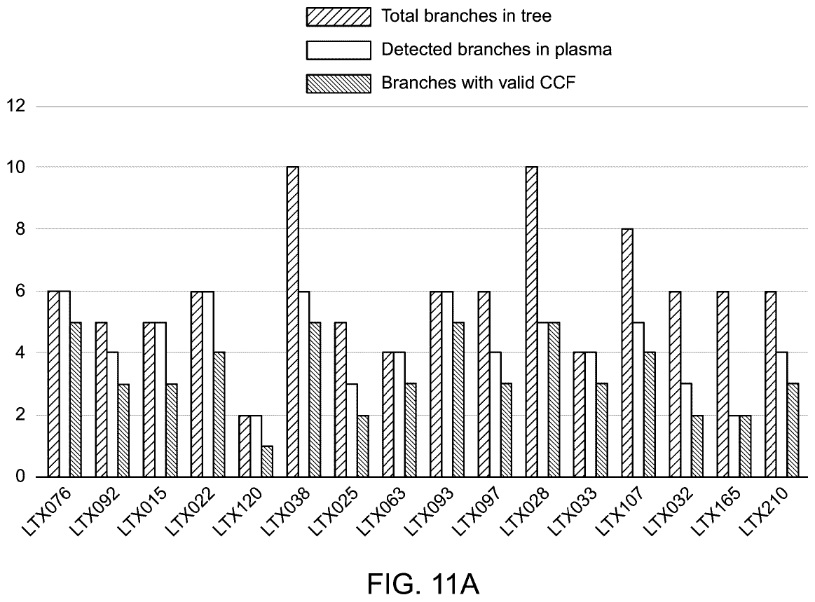

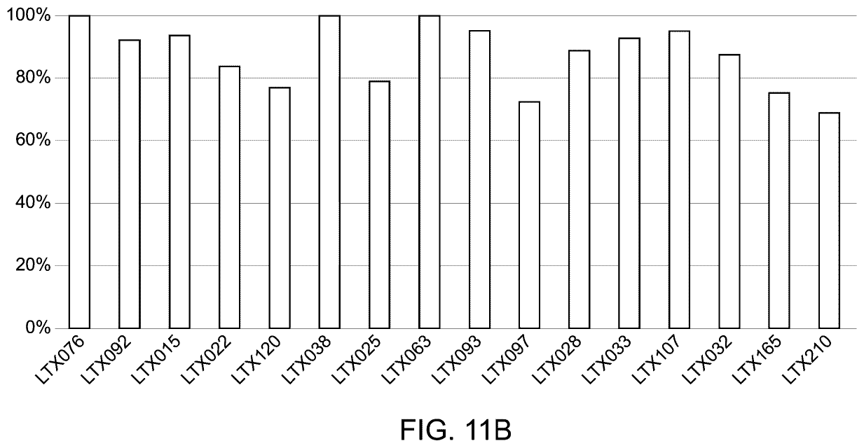

Histological type had the lowest p-value, implying the strongest association, supporting the observation from the sample-level analysis (FIG. 9). The detection rate for SNVs from SQCC was 70% compared with 14% for SNVs from ADC. Mean VAF in tumour was the next significantly associated variable with detection of SNVs in plasma (FIG. 11). Clonal status was also correlated with the detection of SNVs in plasma (detection rate was 48% for clonal SNVs vs 22% for subclonal SNVs). Finally, size of tumour also affected the plasma SNV detection rate (also observed in sample-level analysis).

The remaining variables either did not have statistically significant effects on the detection rate or the supposed effect could be explained by the four variables that were already in the model (for example, tumour VAF is sufficient to explain the differences observed in the detection rate among stages).

### Example 6—Analysis of SNVs not Detected in Plasma

Several lines of evidence support the conclusion that the failure to detect >60% (580 out of 911) of the expected SNVs in the plasma was likely due to those variants not being present in the cfDNA sample, as opposed to some failure of the mPCR/NGS method. We describe these lines of evidence here.
The depth of read (DOR) distribution was similar for the assays that detected the expected plasma SNV and the ones that did not (average DOR 45,551 for assays that detected the expected SNV vs 45,133 for the ones that did not, FIG. 10A). This suggests that assays corresponding to false negative SNV calls were as efficient as those for true positive calls.
Despite the high depth of read at the target SNV position, the number of mutant reads was low for most positions that were not detected: Of 580 SNVs that were not detected, 207 (36%) had 0 mutant reads, and 414 (71%) had <5 mutant reads. Moreover, all of the undetected positions had an observed mutant VAF of less than 0.1%, and the mutation levels in all cases were indistinguishable from their corresponding background error rate.
275 of the 580 SNVs that were not detected belonged to the samples with no plasma mutations, implying that their not being detected could be due to insufficient ctDNA in plasma.
By estimating the limit of detection (LOD) of each position to be 3 standard deviations from the average background mutant VAF at that position, the distributions of the limit of detections for the true-positive and false-negative SNV positions were similar (FIG. 10B). This suggests that if the SNV were present in the sample, it would be detected.

### Example 7—Phylogenetic Trees

To investigate the possibility of being able to recapitulate tumour phylogeny trees from cfDNA data, we examined whether the SNVs detected from plasma were in agreement with the structure of the tumour phylogeny trees reconstructed from tumour tissue data. SNVs detected from plasma can recapitulate tumour phylogeny if they can cover sufficient number of branches in the tree, and their VAF values follow the order of SNVs in the tree—i.e., SNVs located in earlier branches need to have higher VAFs in plasma. More specifically, we define two constraints that should hold for SNVs detected from plasma to be in agreement with the structure of tumour phylogeny tree reconstructed from tissue data:
Constraint 1) For every branch in tree with detected SNV(s) in plasma, the sum of the estimated cancer cell fractions (CCF) of its children branches in tree should not exceed the CCF of that branch
Constraint 2) For every pair of SNVs A and B detected in plasma, if SNV A belongs to a predecessor branch of SNV B, it should have a higher plasma VAF compared with SNV B
   The SNV allele-fraction based on tissue data for each tumour sample was used to reconstruct tumour phylogeny tree using LICHeE. Later, plasma VAF data for detected SNVs was used to evaluate the correctness of constraints 1 and 2 for each sample. To assess the correctness of constraint 1, first we estimated a plasma cancer cell fraction (CCF) for each branch in the tree based on the plasma VAF of detected SNVs in that branch. Then we counted the number of branches in tree with SNVs detected in
   plasma that constraint 1 is valid for them. To assess the correctness of constraint 2, we measured the ratio of all pairs SNVs where one is predecessor of the other and constrain 2 is valid for them.

The results for correlation between plasma SNV data and tumour phylogeny trees for all 49 samples studied here are summarized in Table 7. Since this investigation for samples with very few SNVs detected in plasma is not relevant, in FIGS. 11A and 11B we present our results for samples with at least 10 SNVs detected in plasma. FIG. 11A shows the total number of branches in tree and the number of branches detected in plasma. For 7 out of 17 samples all branches of tree were detected in plasma. FIG. 11A also shows the majority of detected branches have valid CCF estimation meaning constraint 1 is often valid for detected branches. FIG. 11B shows the percentages of SNV pairs that one is predecessor of another in tumour tree and their plasma VAF values confirm the tree structure meaning constraint 2 is valid for them. As it can be seen this percentage is ≥80% for 12 out of 17 samples.

### Example 8—Barcodes

### Background

Through multi-regional whole exome sequencing of resected NSCLC and processing of sequencing data through a bioinformatic pipeline somatic single nucleotide variants (SNVs) are identified and subclonal deconstruction performed to identify whether mutations were subclonal or clonal. Individual mutations are timed as early, late or untimed based on genome doubling events using temporal analysis.

Using “bespoke” variant panels based on barcoding subclonal clusters to track the phylogeny of NSCLC relapse

As in Examples 1 to 7 above, based on multi-regional whole exome sequencing data, subclonal deconstruction and phylogenetic tree analysis, clonal and subclonal variant clusters are identified. Unpublished data shows that NSCLC relapse may be monoclonal and originate from an early truncal clone or specific-subclone, or that relapse may be polyclonal originating from multiple subclones. Through barcoding all variant clusters present in the phylogenetic trees (i.e. having SNVs private to individual clusters represented within a primer assay pool) it has been demonstrated that cfDNA can predict which “part” of the tumour and therefore subclonal population gives rise to the relapse. For example, if the SNVs detected in plasma corresponds to specific subclonal clusters in one branch of the original phylogenetic tree, then it can be inferred that this branch is “seeding” disease relapse. Through using our phylogenetic barcodes, we can track back to the biology of that specific branch and identify therapeutically relevant drivers of relapse that might be used to alter or adapt therapy in the early disease setting. Targeting these drivers of relapse with small molecules or immunotherapies, when disease bulk is low may potentially overcome later problems associated with intratumour heterogeneity driven by the volume of disease and an expanding cancer cell population, that could lead to better patient outcomes.

### Generating Translationally Relevant “High-Sensitivity” Barcodes to Predict Relapse, Minimal Residual Disease, Treatment Responses and Determine the Clonality and Relative Size of Subclones Containing Therapeutically Relevant Variants

As a refinement of the above, targeted sequencing or single-sample whole exome sequencing can be used to generate “early-clonal” barcodes from genomic regions of NSCLC that have been determined to correspond to a high-likelihood of detection in plasma (based on a rational understanding of NSCLC biology). Since early-clonal variants (pre-genome doubling) will be present throughout the NSCLC tumour and amplified due to genome doubling events, a barcode predicted based on these early events before genome doubling will provide maximum sensitivity in terms of a biomarker predictive of NSCLC disease activity and recurrence. This approach will be more clinically applicable due to no pre-requisite requirement for multi-regional sequencing and will have a faster turnaround than the above, therefore being more applicable as a biomarker of recurrence and disease activity in the adjuvant and metastatic disease settings.

To gain maximum sensitivity in identifying minimal residual disease the “early-clonal” barcodes will focus on identifying somatic SNVs in genes classified as being high-confidence early (pre-genome doubling) events in NSCLC carcinogenesis (examples in Table 1) and also identifying somatic SNVs in chromosomal regions classified as being involved in high-confidence early (pre-genome doubling) amplification events (examples in Table 1). Furthermore, mutational signatures may be used to identify variants statistically more likely to be clonal (e.g. signature 4 variants in lung squamous cell carcinoma) (see Table 2).

Once generated this “early-clonal” barcode will be used to generate a bespoke primer panel for that patient based on 30-40 bp amplicon size that covers SNVs determined to be a part of that patient's personal barcode. This primer panel will be used to amplify cell-free DNA libraries. Amplicons will then be sequenced and mutant variants identified. It is envisaged that each panel could then be validated in a pre-surgical plasma sample (e.g. when tumour is in situ). Validated panels will then be used on plasma samples taken 2-6 weeks post-operatively to identify minimal residual disease which will provide prognostic information and potentially identify patients who would benefit from adjuvant chemotherapy. Validated panels will also be used as an inference of tumour bulk and response to adjuvant or palliative chemotherapy. If variant allele frequency of mutants rises during adjuvant or palliative chemotherapy then lack of response could be inferred and treatment could be escalated or altered (to a different treatment modality). If variant allele frequency of mutants falls during adjuvant or palliative chemotherapy then chemotherapy, immunotherapy or targeted therapy doses could be decreased, especially within the context of toxicity. At the point of relapse detection for select patients a primer panel that “barcodes” the subclonal structure of the tumour can be utilised to identify subclonal drivers of relapse that may be amenable to targeted therapy e.g. ERBB2 amplification. Early-clonal barcodes will also be used to monitor immunotherapy, targeted therapy, adoptive cellular therapy, chemotherapy and cancer vaccinations that specifically target the trunk of the tumour.

In the primary and metastatic setting a single biopsy may not provide insight into the breadth of actionable alterations, neo-antigens or potential resistance mechanisms present within the bulk of a patient's disease. Clonal alterations may be lost in a specific tumour region due to copy number events and subclonal alterations may not be represented in a single biopsy. Utilisation of a generic ctDNA driver panel, generic ctDNA resistance mechanism panel or generic ctDNA neoantigen encoding variant panel to probe ctDNA may be performed to identify therapeutically relevant mutations not represented in a single tumour biopsy. The bespoke early-clonal barcode could act as a companion diagnostic tool to determine the therapeutic value of variants not represented in tissue, but identified in plasma by a generic panel. For example, by comparison of the variant allele frequencies of high confidence clonal mutations present in the early-clonal barcode with unknown clonal status mutations identified by generic panels, the clonality of non-tissue represented mutations can be inferred. Furthermore, the cancer cell fraction of a subclonal variant in tissue correlates with the plasma variant allele frequency of said variant. Therefore, the size of a subclone containing a driver, resistance conferring or neoantigen encoding variant, relative to the whole tumour bulk, can be inferred by utilising an early-clonal barcode as a comparison diagnostic tool. This approach may have therapeutic value in identifying and monitoring drivers, resistance mechanisms and neoantigens during treatment and selecting actionable drivers, resistance mechanisms and neoantigens to target with chemotherapy, targeted therapy, dendritic cell vaccines, autologous T Cell therapy and CAR-T cell therapy.

Tracking Clonal SNVs that Encode for Neoantigens in a Patient Specific Barcode

Cytotoxic CD8 and CD4 tumour infiltrating T cells can recognise neoantigens encoded for by tumour SNVs. Clonal neoantigens represent an attractive therapeutic target not confounded by intra tumour heterogeneity (Mcgranahan et al. Science 2016). Adoptive T-cell strategies and cancer vaccinations could be utilised to target clonal neoantigens. Neoantigens can be predicted using bioinformatic approaches. Neoantigen reactive T cells can be identified using MHC multimers that display synthesised peptides encoded for by high confidence neoantigens. Incorporating SNVs that encode for high confidence neoantigens as determined through bioinformatic analysis or neoantigens identified in vitro as being a target for neoantigen reactive T cells, into a patient specific cfDNA barcode, could allow for non-invasive monitoring of adoptive T cell therapy and cancer vaccine approaches through cfDNA genotyping.

### Example 9

### Methods

### Patients

Multiregion tumor samples were collected from 100 treatment naïve patients. Eligible patients were ≥18 years with a diagnosis of stage IA-IIIA NSCLC, with the exception of patient LTX0103 whose tumor was found to be stage IIIB based on post-operative histology. Patients with a malignancy diagnosed or relapsed within 5 years, or those who had received neoadjuvant treatment, were excluded. Histological data were confirmed and standardized by central review. Baseline Positron Emission Topography (PET) CT scans were collected and standardized by central review. Pre-surgical and post-operative plasma samples were collected and processed.

cfDNA Genotyping

Clonal and subclonal SNVs were identified from each patient's baseline exome phylogenetic tree. These SNVs were processed by the standard assay design pipeline and 10 balanced assay pools were created, each containing 10 patients' SNVs and on average 20 assays per patient. Up to 5 mls of plasma was available for each case and timepoint. cfDNA was extracted from plasma and underwent library preparation. Amplified libraries were analyzed by mPCR-NGS. Each PCR assay pool was used to amplify the SNV targets from the 10corresponding samples and 20 negative control samples (plasma libraries prepared from healthy subjects). Each amplicon pool was sequenced on one Illumina HiSeq 2500 Rapid Run with 50 cycles paired-end reads using the Illumina Paired End v1 kit with an average target DOR of ˜40,000 per assay.

### Results

### Patient-Specific Multiplex-PCR Panel Generation

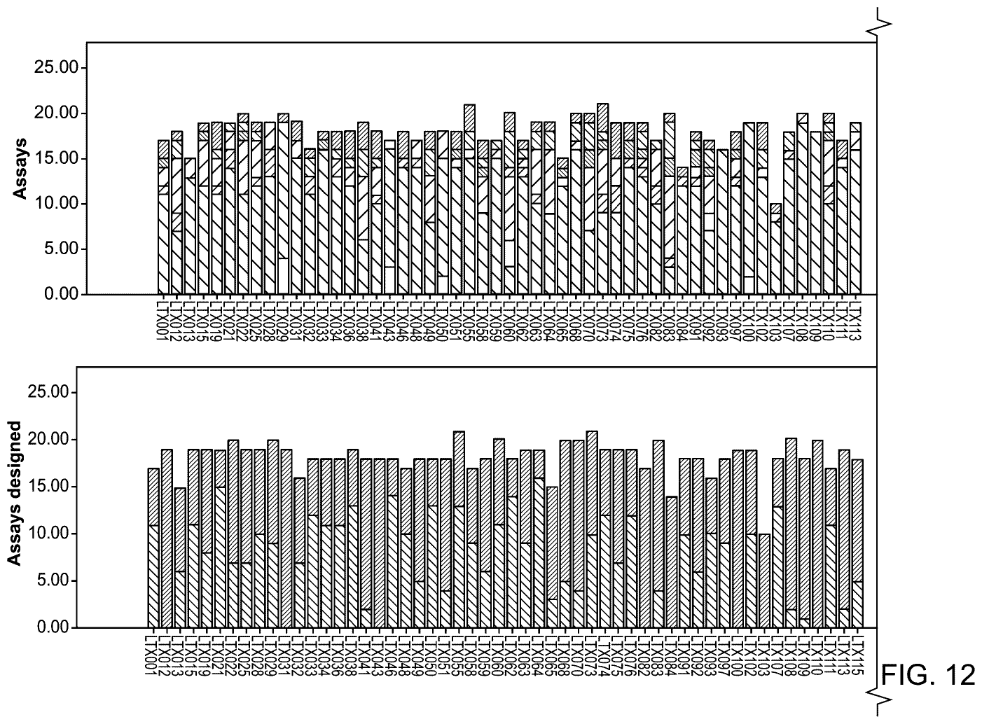

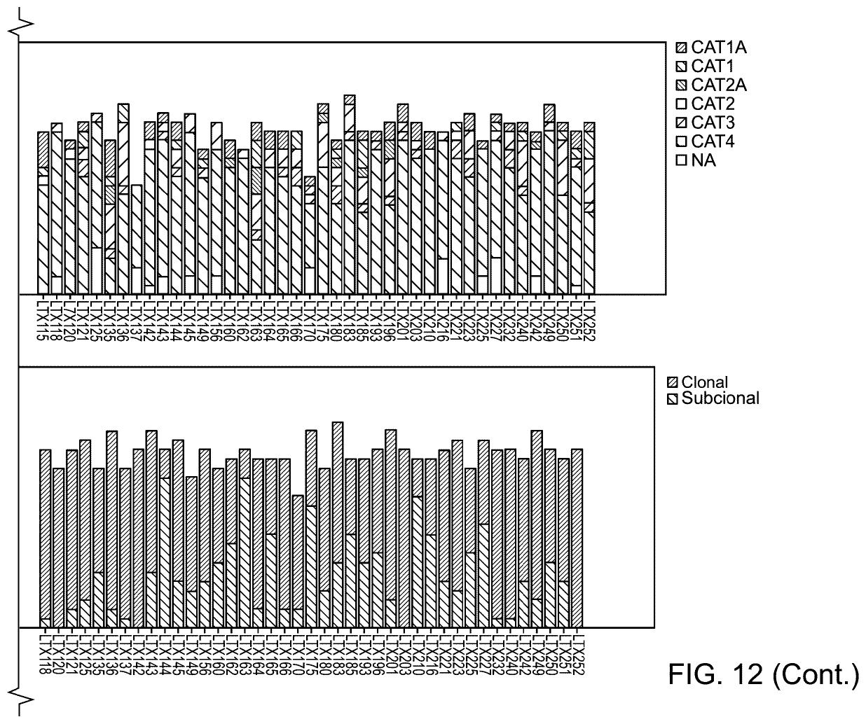

A workflow was designed to facilitate the generation of personalised multiplex-PCR assay pools for the first 100 patients prospectively (FIG. 1). Panels were designed for 96/100 of these patients (FIG. 12). During panel design assay pools were enriched for target driver (category 1a to 2a) variants and designed to ensure uniform sampling of each individual reconstructed tumour phylogenetic tree. The median number of assays within each personalised assay pool was 18 (IQR=2) of these a median of 11 (IQR=8) assays were directed toward truncal variants designated as clonal by exome analysis workflow and a median of 6 (IQR=8) assays were directed toward branch specific variants designated as subclonal, “barcoding” subclonal phylogenetic clusters (FIG. 12). On average 65% of the exome constructed phylogenetic tree was represented by variants targeting one or more subclonal variant clusters. A total of 1748 single nucleotide variants (SNVs) were probed within the baseline pre-surgical cohort and 3172 SNVs were probed in the relapse cohort.

### Distinct Biological Factors Predict Early-Stage NSCLC Detection in Plasma

Pre-operative plasma samples were analysed using the patient-specific multiplex-PCR NGS workflow. A patient's NSCLC was deemed confidently detected if two or more clonal SNVs were identified through profiling of their cfDNA. On this basis 45/96 stage I-IIIA NSCLCs were detected pre-operatively (FIG. 13). Within detected NSCLCs, the median percentage of clonal SNVs identified within assay pools was 94% (IQR=37%), the median percentage of subclonal SNVs identified was 29% (IQR=57%) (FIG. 13). Clonal SNVs were more likely to be identified as present in plasma than subclonal (P<0.001, OR=5.237, [CI 95% 3.9-7.11]).

LUSC histological subtype was observed to be a significant predictor of ctDNA detection (FIG. 13). 30/31 LUSCs were detected, compared with 10/58 LUADs (P<0.001, OR=144 [95% CI 17.5-1182.6]). This observation remained significant in multivariate analysis (MVA) adjusted for composites of pathological TNM stage (tumour volume and nodal involvement), number of clonal mutations in each assay pool and total cfDNA input (ng) (P<0.001, OR=178.3, [95% CI 19.7-1612.1]). Lymph node involvement was also a significant predictor in this model (Table 2). Notably, the multiplex-PCR NGS platform was able to detect 16/17 TNM stage I LUSCs within this cohort compared with only 4/39 stage I LUADs (FIG. 13).

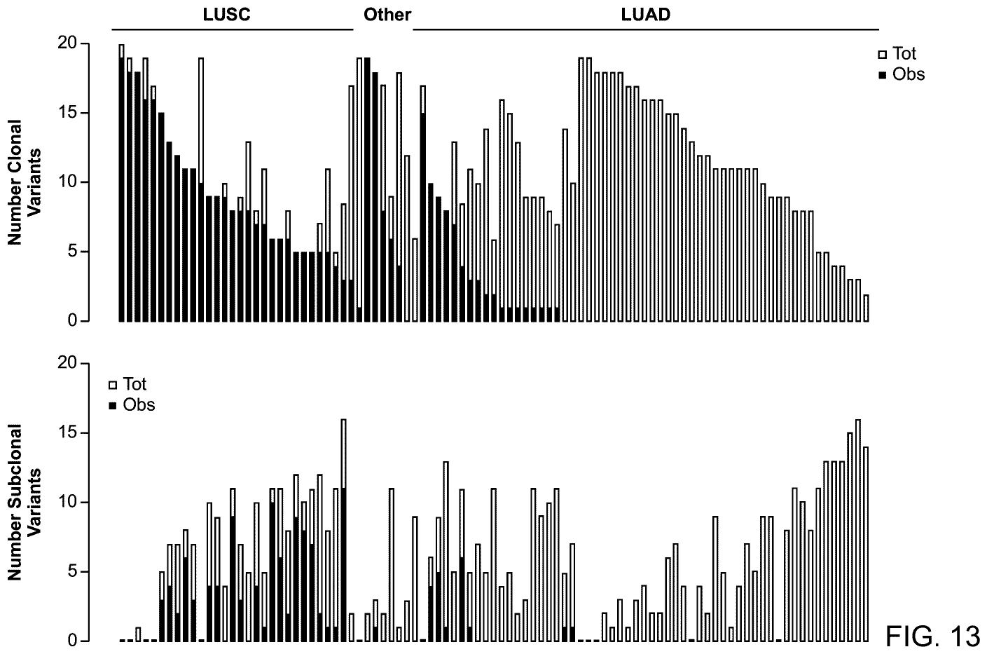

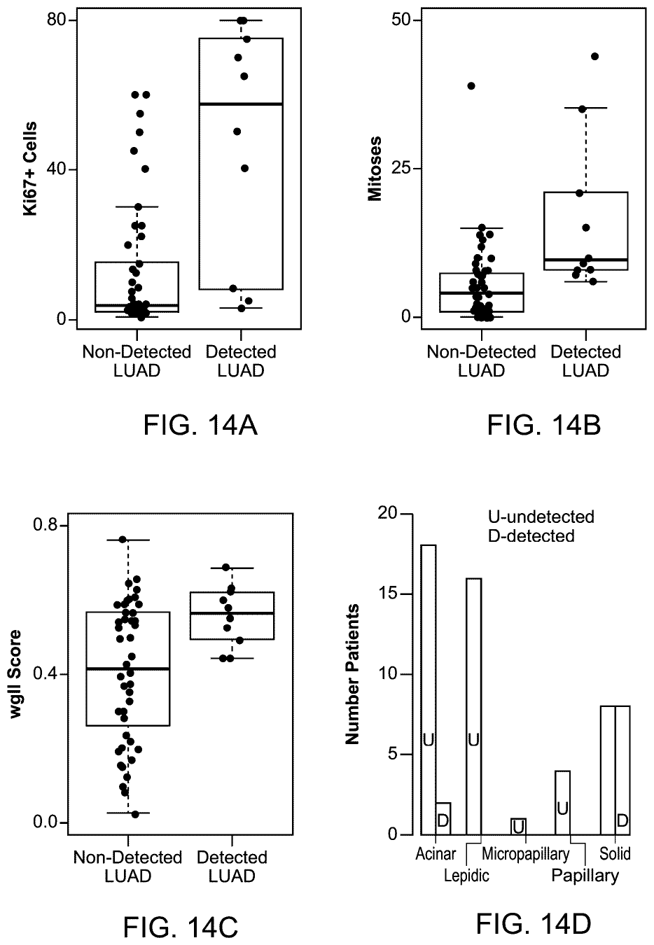

Biological factors that might influence ctDNA release through comparison of features that characterise these histological subtypes were assessed. LUSC, in contrast to LUAD, is a commonly necrotic NSCLC subtype and consistent with this, LUSCs present within the cohort were more necrotic than LUADs as determined by histological examination (median necrosis 40% versus 2%, Mann-Whitney U Test P<0.001). Consistent with the importance of necrosis, detected LUADs were significantly more necrotic than undetected LUADs (median necrosis 17.5% versus 2%, Mann-Whitney U Test P=0.004). Similar to previous studies of early-stage NSCLC, LUSCs were more PET avid than LUADs (median Tumour Background Ratio (TBR) 9.0 versus 4.6 Mann-Whitney U Test P<0.001) (8, 9) and PET avidity was significantly higher in detected LUADs compared with non-detected LUADs (median TBR 10.2 compared with 3.6, Mann-Whitney U Test P=0.001). Both necrosis and PET avidity were the only significant predictors of LUAD detection in a sub-group MVA performed to ascertain the effects of necrosis, PET TBR, lymph node involvement, tumour volume, number of clonal mutations in each assay pool and total cfDNA input (ng) (Table 3). Since PET avidity in stage I NSCLC correlates with tumour doubling time and hypoxic necrosis occurs in rapid proliferating tumours, we hypothesised that high cell turnover could contribute to these observations (10). Ki67 immunohistochemistry performed on a tissue microarrays of the cohort revealed a higher number of Ki67 positive cells in LUSCs compared with LUADs (median percent of Ki67+ cells 50% in LUSCs versus 4% in LUAD, Mann-Whitney U Test P<0.0001) and detected LUADs demonstrated significantly higher Ki67 positivity than non-detected LUADs (median percent of Ki67+ cells 65% in detected LUADs versus 3% in non-detected LUADs, Mann-Whitney U Test P=0.007) (FIG. 14). Detected LUADs were also observed to have a significantly higher number of peripheral mitotic figures per high power field (HPF) than undetected LUADs on blinded histological review (Mann-Whitney U Test P<0.001) (FIG. 14) Taken together, these data suggest that biological factors governing tumour specific cell cycle kinetics inherent to LUSCs, present in a sub-population of LUADs governs ctDNA release, rather than TNM stage or technical factors.

Notably 8/10 detected LUADs demonstrated solid predominant histology, a LUAD subtype associated with higher rates of proliferation and a worse prognosis, no lepidic predominant LUADs were detected (FIG. 14).

Variant Allele Frequency is Associated with Tumour Size, Clonality and Copy Number Status

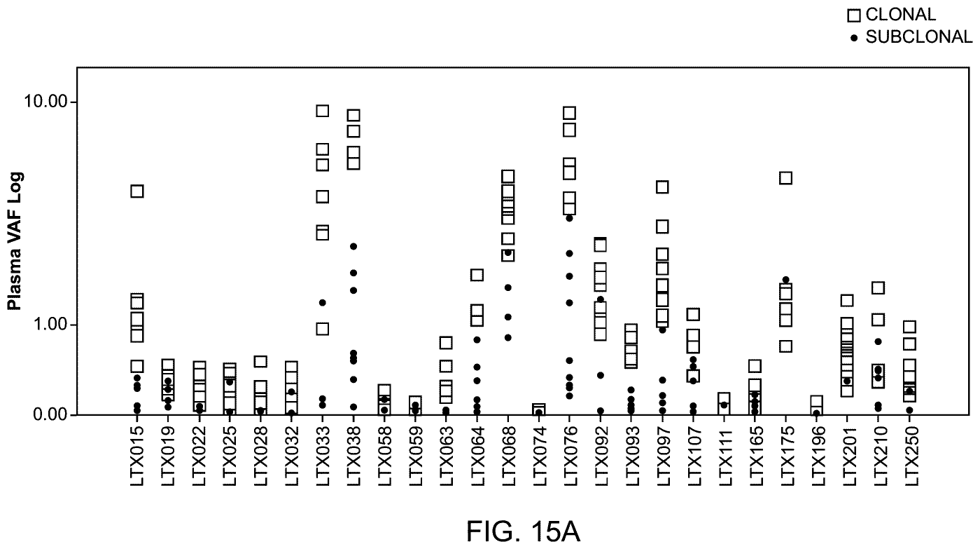

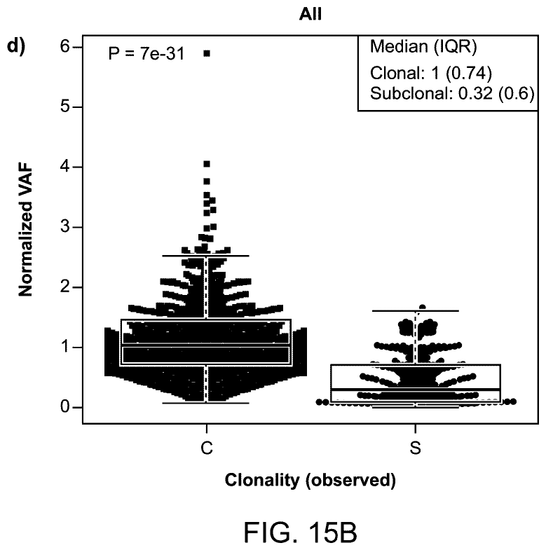

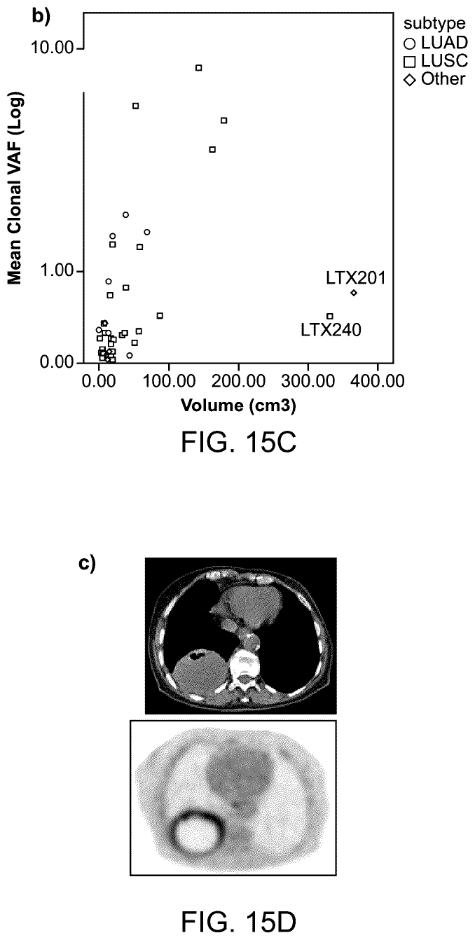

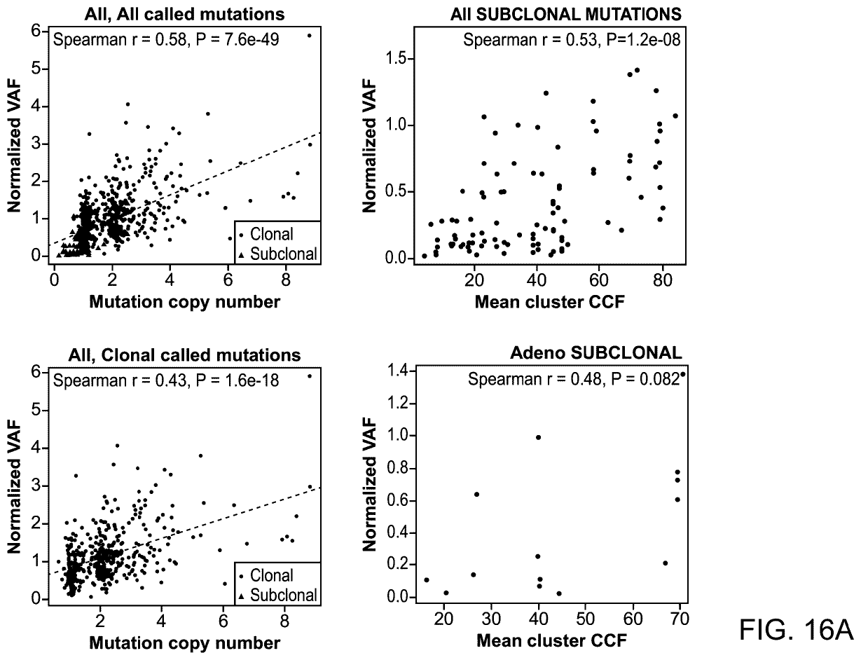

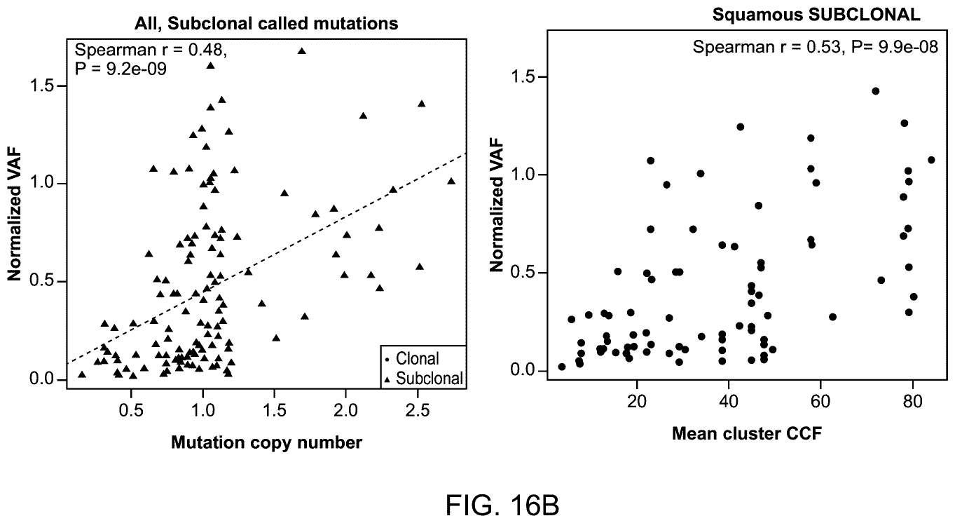

Factors involved in the frequency of variant detection within the plasma of the confidently detected patients with confidently detected tumours was determined. Variant allele frequencies (VAFs) of detected mutations showed significant inter-patient heterogeneity (FIG. 15A). Mean clonal VAF significantly correlated strongly with tumour volume (Spearman's Rank Rₛ(42)=0.60, P<0.001) (FIG. 15C); LTX201 and LTX240 were outliers within the context of this observation. LTX201 is an adeno-squamous carcinoma and was determined to be predominantly LUAD on histological review. Given our previous observations regarding limited ctDNA detection from LUADs this raises the possibility that only the LUSC component of this tumour was releasing ctDNA. LTX240 was a large tumour that on radiological review was noted to consist of a rim of PET avid tumour surrounding a large necrotic cavity, highlighting limited viable tumour volume (FIG. 15D). There was no observed association between percentage of necrosis on histological examination, lymph node involvement, PET TBR, histological subtype, cfDNA input quantity and tumour location (central, peripheral, upper/middle or lower lobe) with mean clonal VAF in univariate analyses. Normalisation of VAF was performed to control for the effect of tumour size, we noted that SNVs designated as clonal had significantly higher normalised VAFs than SNVs designated as subclonal (median VAF for clonal SNVs 1±0.74 versus 0.32±0.6 for subclonal SNVs, Mann-Whitney U Test P<0.001) (FIG. 15B). Notably the mean Cancer Cell Fraction of a detected subclonal variant across regions sampled for m-seq correlated with normalized plasma VAF suggesting that subclone size can be inferred from plasma VAF and we observed that the copy number status of detected SNVs correlated with normalised plasma VAF (FIG. 16).

Given the observed difference in VAF between clonal and subclonal mutations whether plasma genotyping could act as a companion diagnostic tool to a single-region biopsy in order to infer clonality of detected driver variants was determined.

### CIDNA Detection Heralds Relapse in the Adjuvant Setting

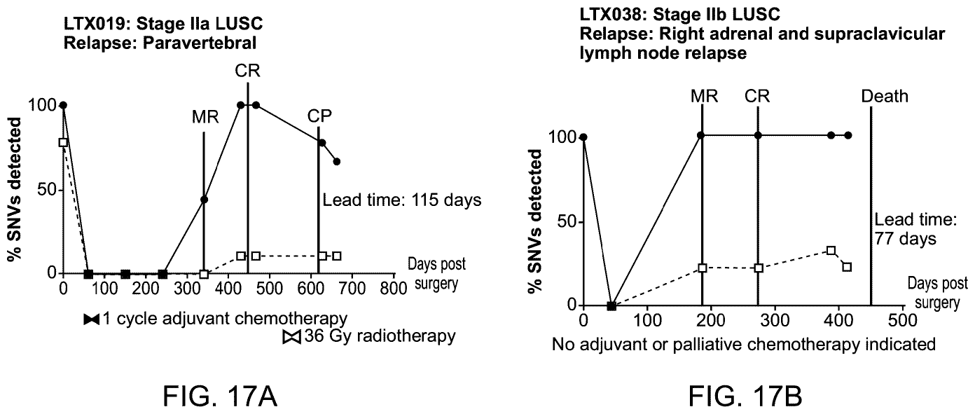

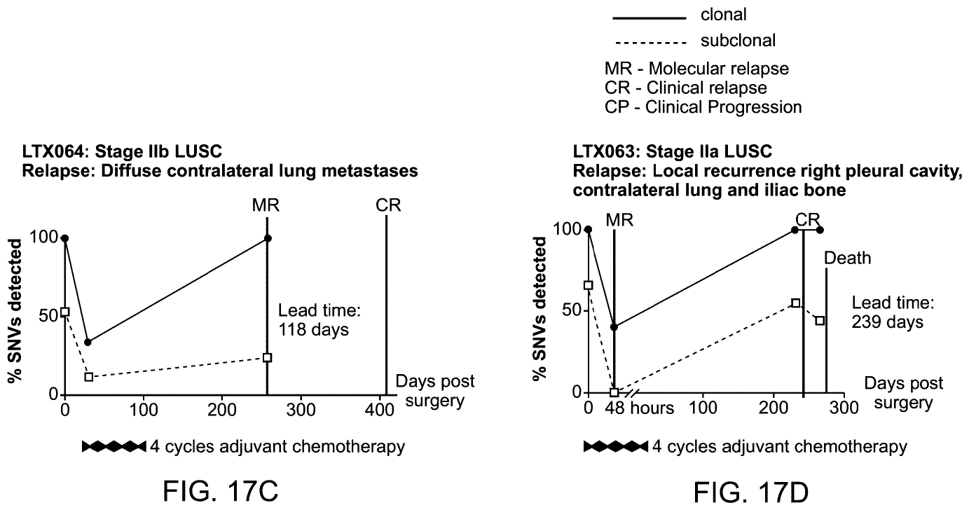

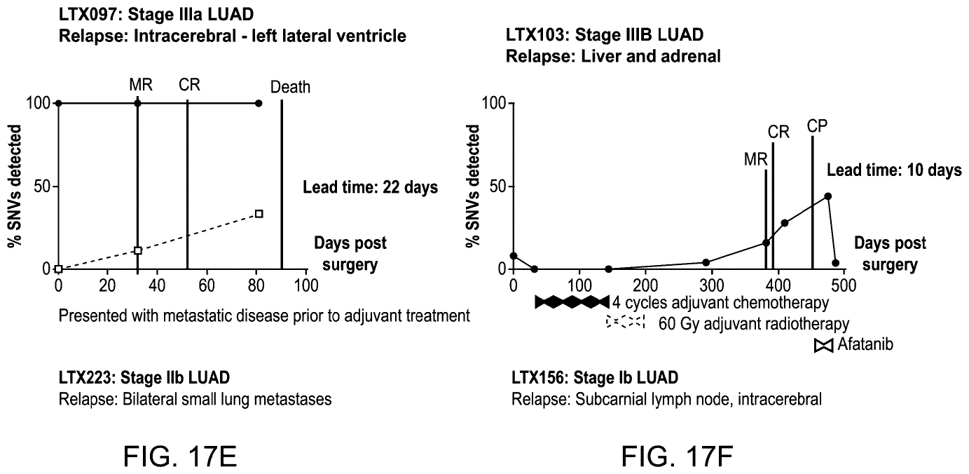

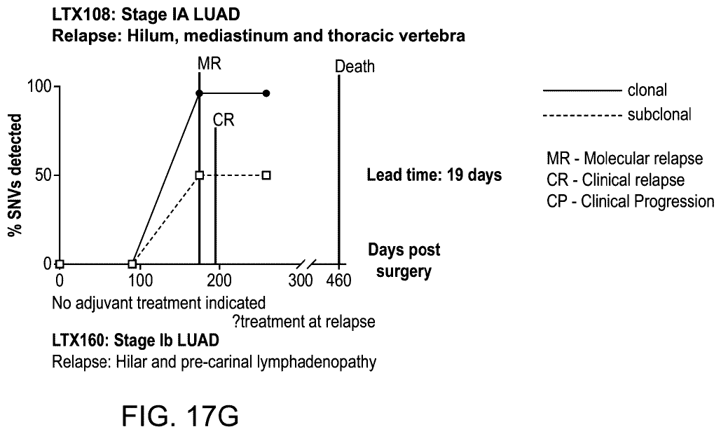

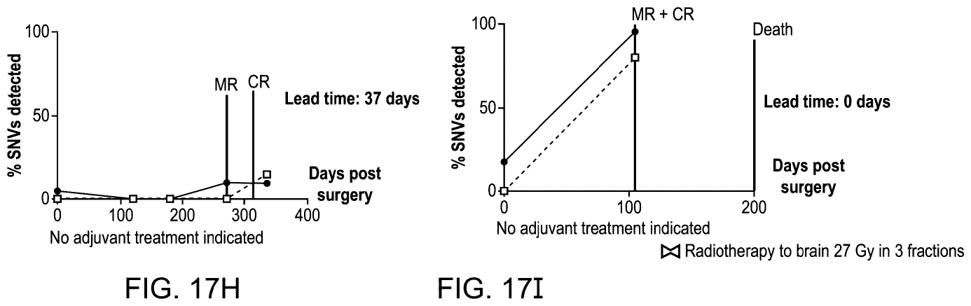

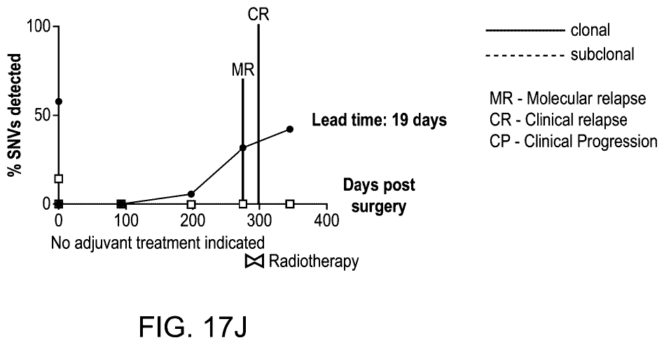

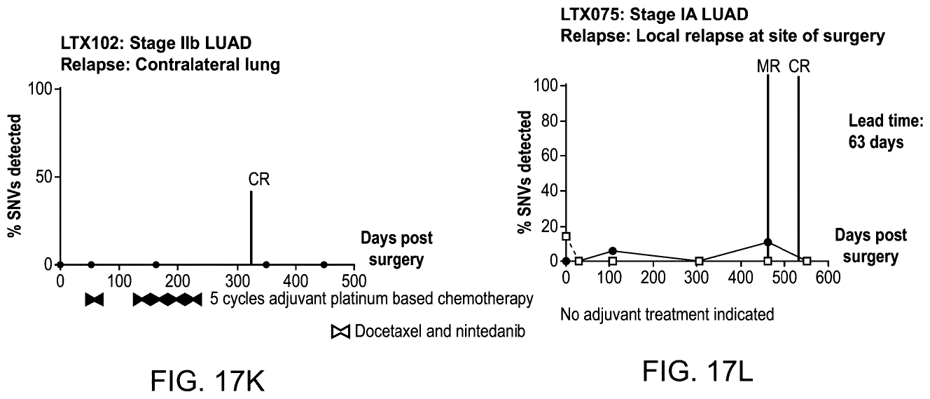

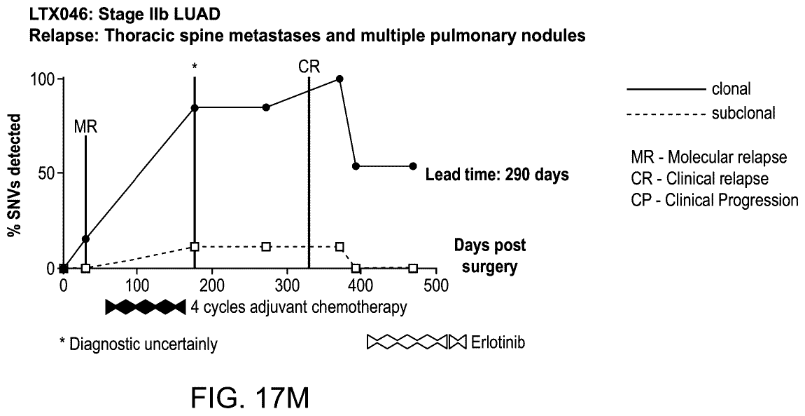

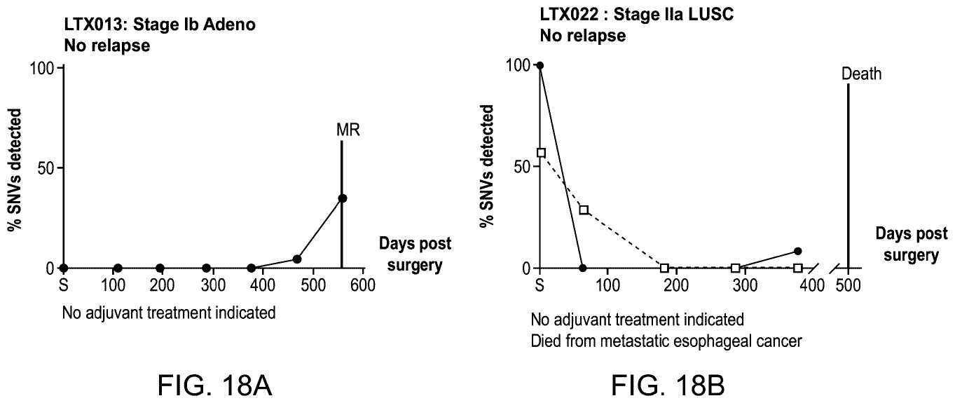

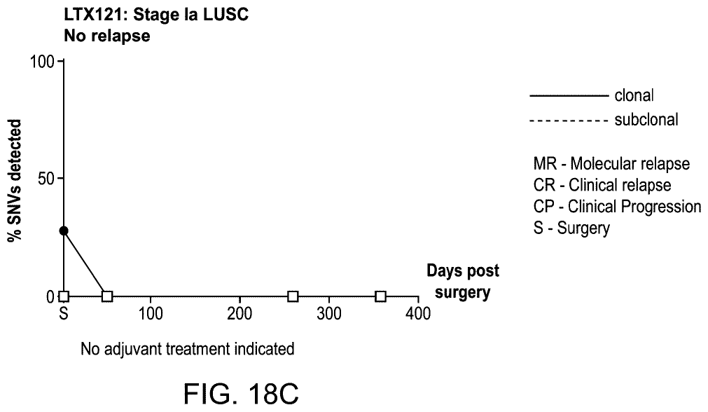

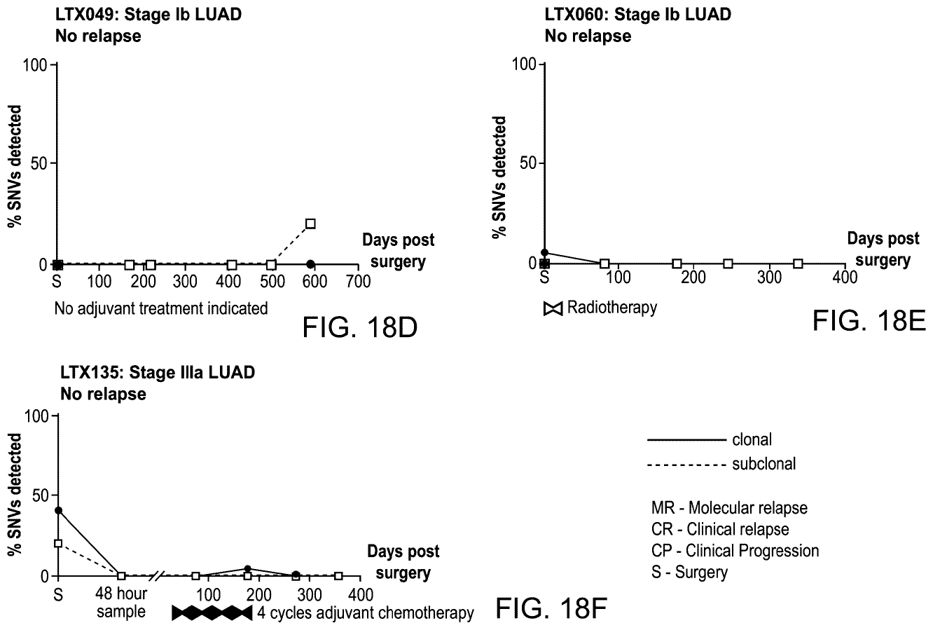

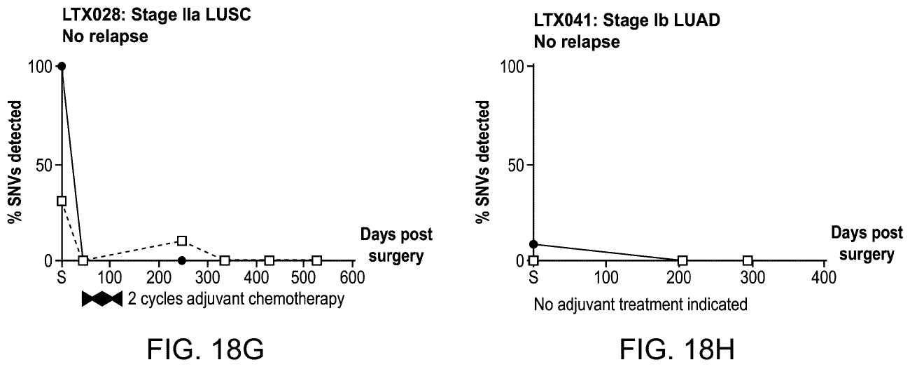

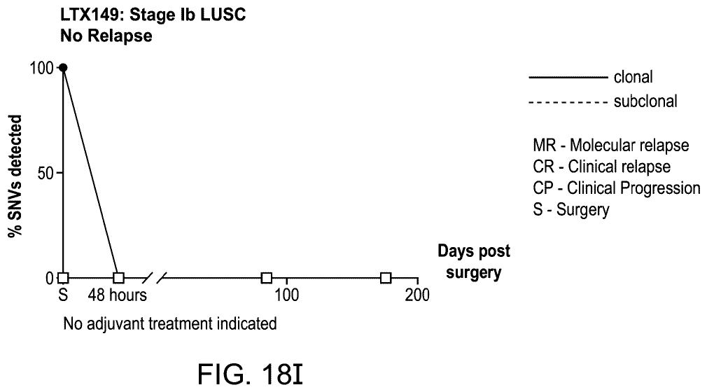

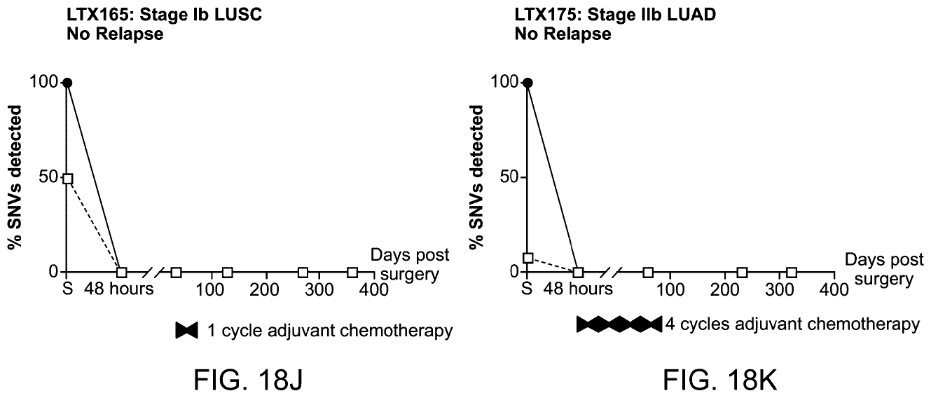

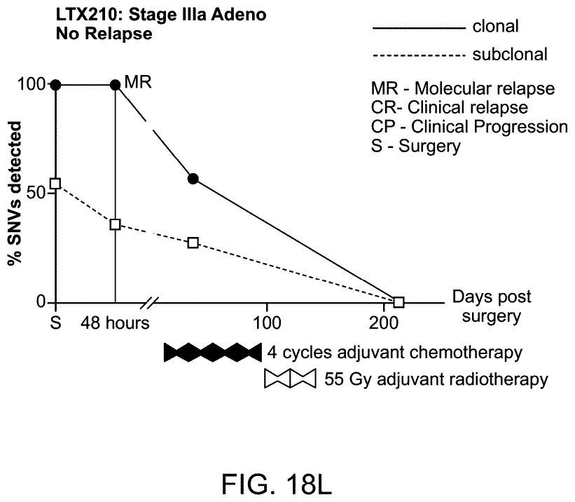

A recurrence sub-cohort of 25 patients (13 patients whom within this 96 patient cohort suffered clinical recurrence of their NSCLC within the time-frame of this study and 12 controls). Plasma samples from this cohort donated at regular intervals within the post-operative setting were subject to cfDNA genotyping in a blinded fashion. 12 out of 13 NSCLC relapses were detected in plasma (FIG. 17). The undetected relapse case (LTX102) was notably a lepidic predominant LUAD with a low wgII score, in our baseline data we noted that ctDNA was unable to detect tumours with similar characteristics. Defining molecular relapse as the detection of two clonal SNVs in the post-operative setting achieved the highest sensitivity and specificity in terms of identifying relapse (92% and 83% respectively) as compared with utilising other SNV thresholds (Table 10). Notably two clonal SNVs were detected in the plasma of two control cases: LTX013 and LTX210. The detection of an increasing number of tumour specific SNVs, on two occasions in the plasma of LTX013 is concerning for sub-clinical progression that could potentially manifest clinically with longer follow-up (FIG. 18A). LTX210 demonstrated evidence of minimal residual disease with tumour specific SNVs detectable at 48 hours and 38 days after surgery. Following commencement of adjuvant chemotherapy and radiotherapy evidence of complete molecular response was observed (FIG. 18L). No evidence of molecular relapse was observed in the remaining 10 control cases (FIG. 18).

The median interval between the detection of molecular relapse and clinical relapse (lead time) was significantly longer in LUSCs compared with LUADs (median lead time in LUSCs 116.5 days (IQR=122.25, n=4) versus LUADs 20.5 days (IQR=44.25, n=8), Mann-Whitney U Test P=0.048). Notably the outlier for the LUAD cohort was LTX046, which was detected in plasma 290 days prior to the confirmation of clinical relapse. In this case a CT Thorax and Abdomen had been performed 133 days prior to confirmed relapse, for surveillance of a lung nodule (annotated in FIG. 17M). This CT had demonstrated an equivocal sclerotic focus in a thoracic vertebra and an interval CT scan to monitor this lesion was arranged. Companion cfDNA genotyping at this time point could have supported clinical relapse, perhaps leading to earlier initiation of anti-EGFR monotherapy.

Tracking Tumour Evolution Through Plasma cfDNA Genotyping

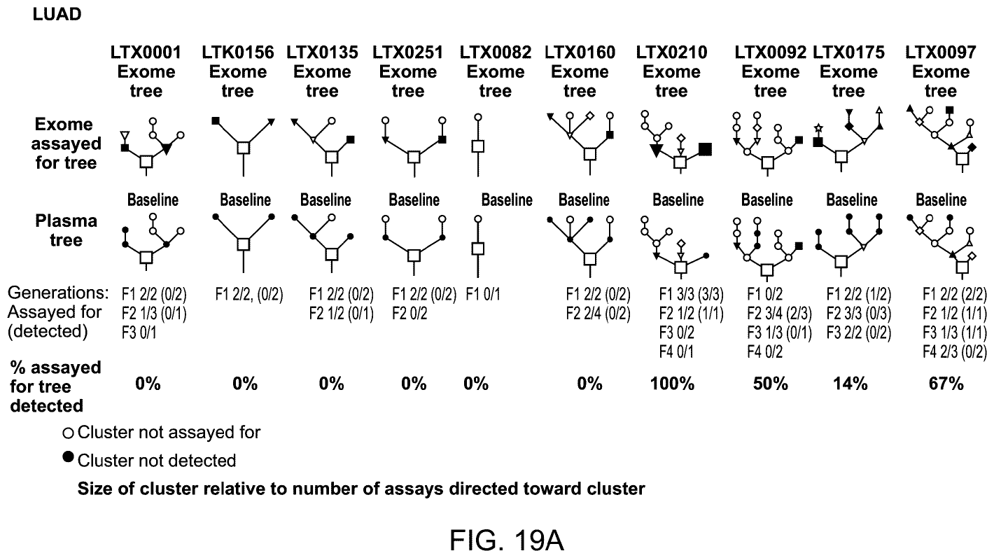

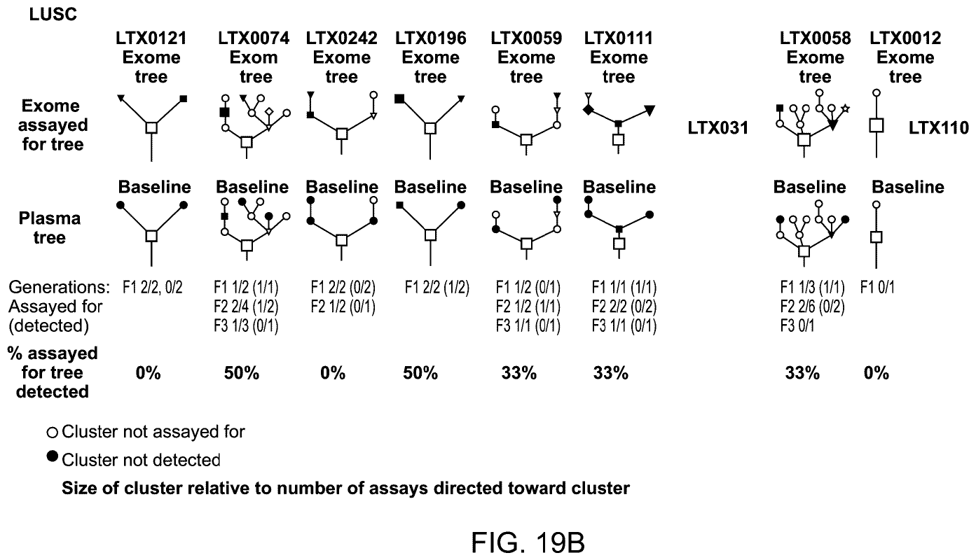

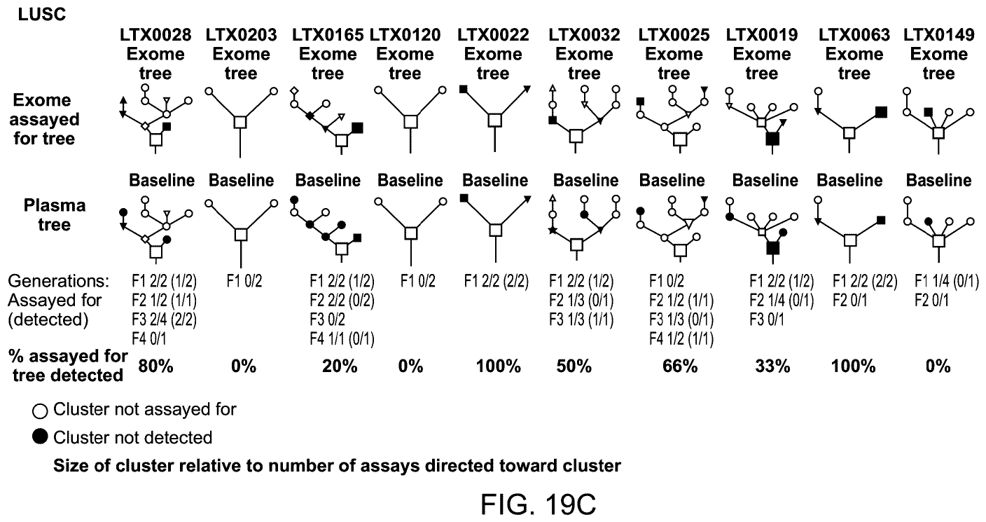

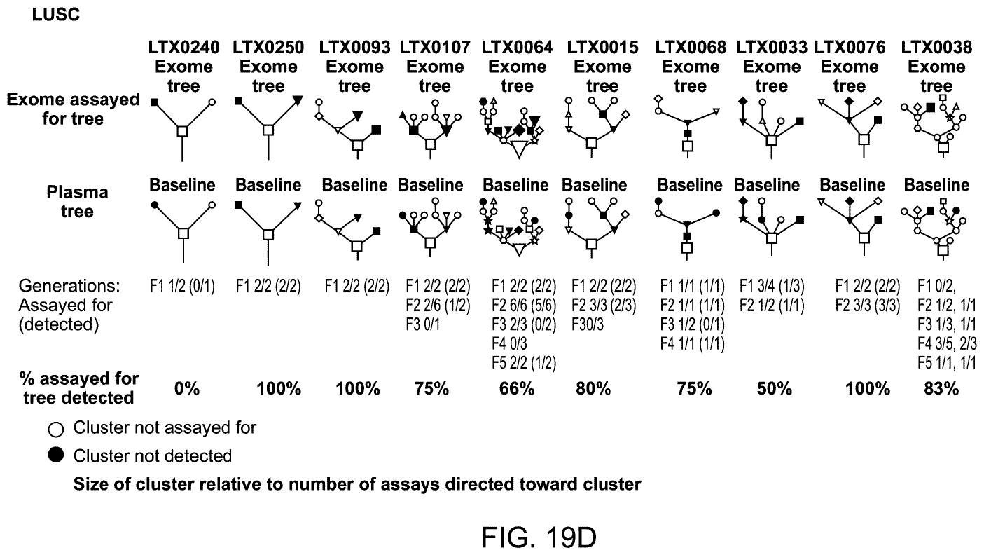

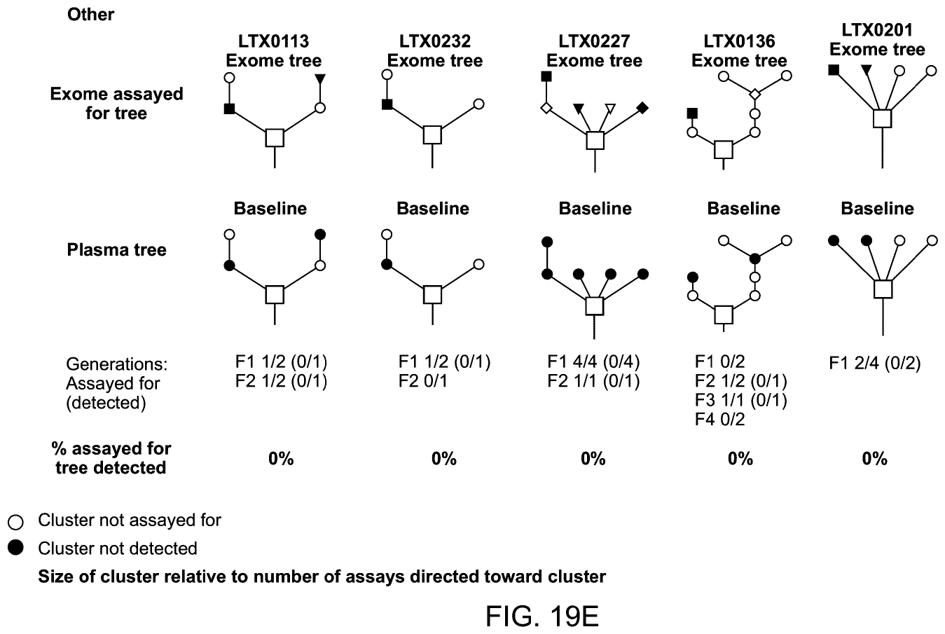

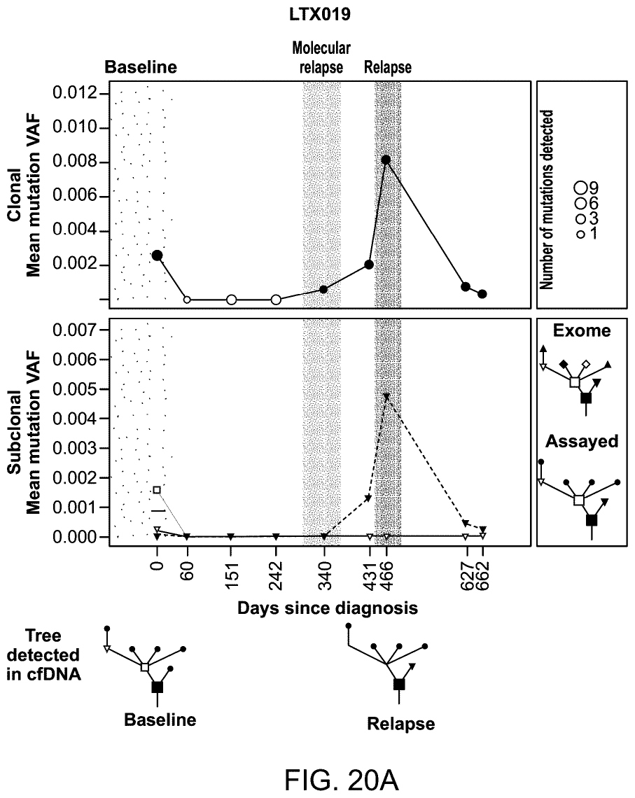

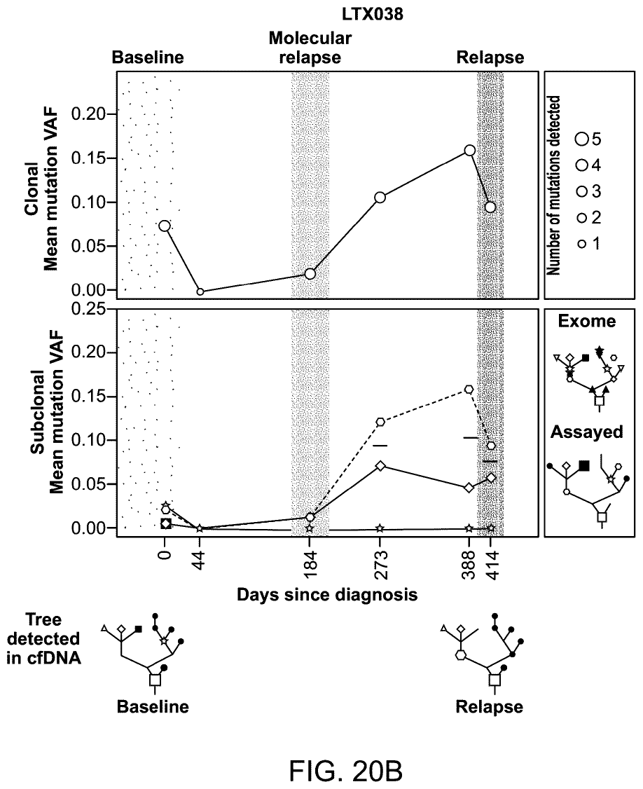

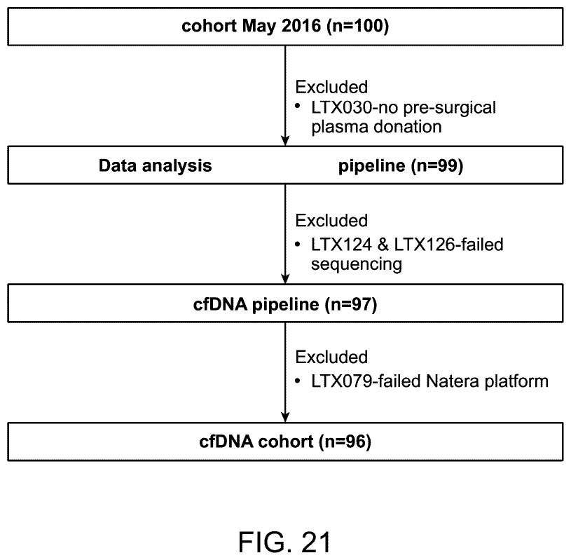

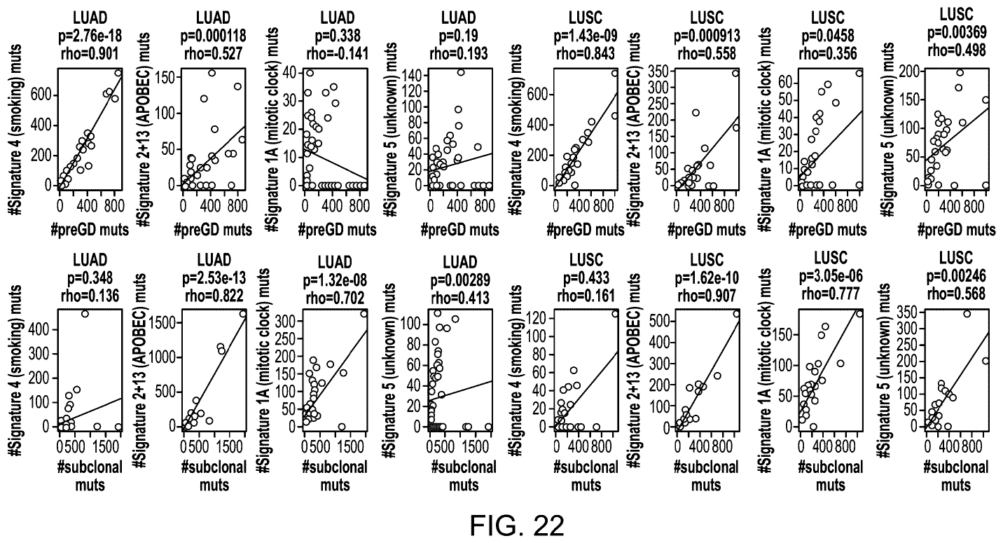

Subclonal node “barcoding” strategy used to non-invasively monitor NSCLC evolutionary dynamics occurring at the point of disease relapse was determined. In pre-surgical plasma samples a mean percentage of 55% (SD±35%, range 0-100%) barcoded subclonal clusters were represented in the plasma of detected LUSCs and a mean percentage of 26% (SD±37%, range 0-100%) barcoded subclonal nodes were represented in the plasma of detected LUADs suggesting that cfDNA genotyping can be utilised to monitor tumour phylogenetic structure in plasma (FIG. 19). In LTX019 two F1 subclonal nodes (defined as subclonal nodes sharing the clonal node as their most recent ancestor) were barcoded in the assay pool. Only one of these nodes was represented in the pre-surgical plasma suggesting that this was the dominant branch of the phylogenetic tree in the primary tumour. At relapse the undetected F1 subclonal node was pervasive in plasma (FIG. 20). Interestingly, exome analysis of the primary tumour revealed this node contained a subclone specific amplification in 11p13 involving CD44, a lymphocyte homing receptor, thought to be involved in preparation of the pre-metastatic niche. In LTX038, both branches of the barcoded exome phylogenetic tree were detectable at baseline in plasma. At relapse, only a single branch was detectable and variants barcoding subclonal nodes on this branch were detected at significantly higher frequencies in plasma. Notably an F4 subclonal node that enriched in plasma at the point of relapse contained a Notch1 driver mutation (FIG. 20). Unfortunately, this patient declined rapidly following their relapse and tissue from this site was not available for exome analysis.

Lengthy table referenced here
US20260022422A1-20260122-T00001
Please refer to the end of the specification for access instructions.

Lengthy table referenced here
US20260022422A1-20260122-T00002
Please refer to the end of the specification for access instructions.

Lengthy table referenced here
US20260022422A1-20260122-T00003
Please refer to the end of the specification for access instructions.

Lengthy table referenced here
US20260022422A1-20260122-T00004
Please refer to the end of the specification for access instructions.

Lengthy table referenced here
US20260022422A1-20260122-T00005
Please refer to the end of the specification for access instructions.

Lengthy table referenced here
US20260022422A1-20260122-T00006
Please refer to the end of the specification for access instructions.

Lengthy table referenced here
US20260022422A1-20260122-T00007
Please refer to the end of the specification for access instructions.

Lengthy table referenced here
US20260022422A1-20260122-T00008
Please refer to the end of the specification for access instructions.

Lengthy table referenced here
US20260022422A1-20260122-T00009
Please refer to the end of the specification for access instructions.

Lengthy table referenced here
US20260022422A1-20260122-T00010
Please refer to the end of the specification for access instructions.

Lengthy table referenced here
US20260022422A1-20260122-T00011
Please refer to the end of the specification for access instructions.

Lengthy table referenced here
US20260022422A1-20260122-T00012
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260022422A1). An electronic copy of the table will also be available from the USPTO upon request and payment of the fee set forth in 37 CFR 1.19(b)(3).

1. A subject-specific method for detecting recurrence of a tumour in a subject, comprising:
a) sequencing all or part of the genome or exome of a tumour of a subject to define clonal and/or subclonal mutations in said tumour;
b) defining a set of reagents that will detect the presence of DNA from said tumour via the presence of said clonal and/or subclonal mutations; and
c) using said set of reagents, analysing a sample comprising DNA from said tumour obtained from the subject subsequent to the tumour removal to determine whether or not said tumour has recurred by detection of said clonal and/or subclonal mutations in the sample.

2. A subject-specific method of defining a set of reagents to detect recurrence of a tumour in the subject comprising:
a) sequencing all or part of the genome or exome of a tumour of the subject;
b) defining clonal and/or subclonal mutations in said tumour; and
c) defining a set of reagents that will detect the presence of said clonal and/or subclonal mutations in a sample from said subject.

3. A subject-specific method for detecting recurrence of a tumour in a subject comprising: analysing, with a subject-specific set of reagents that will detect the presence of clonal and/or subclonal mutations from said tumour, DNA in a sample obtained from the subject subsequent to removal of a tumour to determine whether or not said tumour has recurred by detection of said clonal and/or subclonal mutations from the tumour in the sample.

4. The method of claim 1, wherein said sequencing is carried out on a tumour biopsy, all or part of the tumour or one or more subsections of the tumour, or on cell free DNA (cfDNA), circulating tumour DNA, exosome derived tumour DNA or circulating tumour cells from the subject.

5. The method of claim 4, wherein said sequencing is carried out on said tumour or subsection thereof following removal of the tumour.

6. The method of claim 1, wherein the sample to be analysed comprises cfDNA, circulating tumour DNA, exosome derived tumour DNA or circulating tumour cells.

7. The method of claim 1, wherein all or part of the genome or exome of at least two subsections of the tumour is sequenced and at least one of clonal and subclonal mutations are defined based on which mutations occur in which tumour subsections.

8. The method of claim 7, wherein the at least one of clonal and subclonal mutations in the tumour are defined by sequencing the genome or exome of up to ten subsections of the tumour.

9. The method of claim 8, wherein the at least one of clonal and subclonal mutations are single nucleotide variant (SNV) mutations.

10. The method of claim 9, wherein said set of reagents is a set of multiplex PCR primers and said analysis is a multiplex PCR.

11. The method of claim 10, further comprising synthesising said set of reagents.

12. The method of claim 1, further comprising sequencing all or part of the non-tumour genome or exome of the subject and comparing this with said tumour genome or exome sequences to define the clonal/sub clonal mutations found in the subject's tumour, wherein said set of detection reagents detects at least one or at least two clonal mutations and said set of detection reagents detects at least one mutation characteristic of each subsection of the tumour.

13. (canceled)

14. (canceled)

15. The method of claim 1, wherein clonal and subclonal mutations are assembled into a phylogenetic tree defining the clonal/subclonal mutation profile of the tumour and wherein said set of detection reagents is capable of detecting at least one mutation characteristic of the trunk of the phylogenetic tree and at least one mutation characteristic of each branch of the phylogenetic tree.

16. (canceled)

17. The method of claim 1, wherein the whole genome or exome of said DNA from said tumour and/or the whole exome of said at least two tumour subsections and/or the whole non-tumour exome of the subject is sequenced.

18. The method of claim 1, wherein part of the genome or exome is sequenced to identify early clonal mutations from the subject's tumour in the subject's cfDNA and wherein the early clonal mutations are somatic SNVs in genes classified as being high-confidence pre-genome doubling events and/or somatic SNVs in chromosomal regions classified as being involved in high-confidence early (pre-genome doubling) amplification events.

19. (canceled)

20. (canceled)

21. The method of claim 1, wherein said tumour is at least one of a solid tumour, a lung tumour, a breast tumour, a colorectal tumour, a prostate tumour or a melanoma, and wherein the lung tumour is a non-small cell lung tumour, and further wherein the non-small cell lung tumour is a squamous cell carcinoma, an adenocarcinoma or a large cell carcinoma.

22. (canceled)

23. (canceled)

24. (canceled)

25. The method of claim 1, wherein the said sequencing is carried out on blood plasma obtained from the subject, or said sample to be analysed is a blood plasma sample from the subject.

26. The method of claim 25, wherein the analysis is carried out at least 48 hours, from 48 hours to 7 days, from 7 to 14 days, from 14 days to one month, from one to two months, from two to six months, from six months to one year or over one year from the removal of the tumour.

27. (canceled)

28. The method of claim 7, wherein said subject is a human subject and further comprising: selecting a treatment for the patient based on the at least one of clonal and subclonal mutations detected in the sample from the subject and administering the treatment to the subject, optionally the early clonal mutations are detected in one or more of the genes of Table 1.

29. (canceled)

30. (canceled)

31. (canceled)

32. (canceled)

33. The method of claim 28, wherein the treatment is an adjuvant therapy.
